(19) United States
(12) Patent Application Publication
(10) Pub. No.: US 2025/0040558 A1
(43) Pub. Date: Feb. 6, 2025

(54) ANTIMICROBIAL ACTIVITY OF CHLORITE-ENTRAINED POLYMERS

(71) Applicant: CSP Technologies, Inc., Auburn, AL (US)

(72) Inventors: Angela Morgan, Auburn, AL (US); Jason Pratt, Auburn, AL (US); Michael A. Johnston, Atlanta, GA (US); John Belfance, Auburn, AL (US); Ivy Comer, Auburn, AL (US); Autumn Reynolds, Auburn, AL (US)

(21) Appl. No.: 18/704,982

(22) PCT Filed: Oct. 26, 2022
(86) PCT No.: PCT/US2022/078728

§ 371 Date: Apr. 26, 2024

Related U.S. Application Data
(60) Provisional application No. 63/263,062, filed on Oct. 26, 2021.

(51) Int. Cl. A23B 7/152; A23B 7/055; C01B 11/02

(52) U.S. Cl. CPC A23B 7/152; A23B 7/055; C01B 11/024

(57) ABSTRACT

This disclosure relates to a composition, a system and a method of reducing and preventing the growth of microbes, or for killing microbes. More particularly, the disclosure relates to a composition, a system and a method of reducing and preventing growth of microbes, or for killing microbes, e.g., in food and medical equipment containers, using polymers entrained with antimicrobial agents.

This application claims priority under 35 U.S.C. § 119(e) from U.S. Provisional Patent Application No. 63/263,062, entitled “USE OF CHLORITE-ENTRAINED POLYMERS FOR INHIBITING VIRUSES ON FOODS”, filed on Oct. 26, 2021, the contents of which are incorporated herein by reference in their entirety.

### FIELD OF THE INVENTION

This disclosure relates to a composition, a system and a method of reducing and preventing the growth of microbes, or for killing microbes. More particularly, the disclosure relates to a composition, a system and a method of reducing and preventing growth of microbes, or for killing microbes, e.g., in food and medical equipment containers, using polymers entrained with antimicrobial agents.

### BACKGROUND OF THE INVENTION

There are many items that are preferably stored, shipped and/or utilized in an environment that must be controlled and/or regulated. For example, in the moisture control field, containers and/or packages having the ability to absorb excess moisture trapped therein have been recognized as desirable. Likewise, in packaging products that carry a risk of contamination, e.g., food or medical equipment, it may be desirable to control the growth and proliferation of microbes.

Foodborne illness, such as food poisoning from Salmonella or E. coli bacteria, is common, costly, and preventable. Although the presence of Salmonella and E. coli in a variety of raw ingredients is well understood, many are unaware of the risks of these microorganisms in one of the most common ingredients of all: flour. The CDC estimates that each year 1 in 6 Americans get sick from contaminated food or beverages, and 3000 die from foodborne illness. The U.S. Department of Agriculture estimates that foodborne illnesses cost the United States more than $15.6 billion each year.

In addition to foodborne illnesses caused by bacteria, viral contamination of food can also cause illnesses. Among the most prominent viruses that contaminate food are human norovirus (HNV) and hepatitis A (HAV). Viral contamination is more difficult to assay than bacterial contamination since viruses are difficult to culture, can produce mild symptoms that can be problematic to quantify, and limited global pathogenic viral surveillance systems are in place globally. For these reasons, the prominence of virally mediated foodborne diseases is likely to increase as diagnostic methods and surveillance systems improve over time.

One method that the food industry has employed for preservation of foodstuffs is by including food grade preservatives, such as potassium sorbate, sodium benzoate and nitrites, as a component of the food. However, such preservatives are regarded by some in the health field and consumers as being unnatural and presenting health risks. Moreover, it is not practical to use such preservatives with non-processed foods, for example, fresh fruits or vegetables.

Another method for reducing microbes in foodstuffs is exposure to heat. Cooking foodstuffs can be an option for combating microbial contamination. In certain cases, this approach can be the only available option, since certain foodborne bacteria, specifically Listeria monocytogenes, can only be eradicated by heating the food to a temperature above 74° C. or 165° F. In theory, fruits and vegetables can be heat processed prior to freezing; however, in practice, this heat processing can substantially degrade the appeal to the consumer for these foods. Not only can organoleptic qualities such as flavor be affected by cooking, but suitability for use in food preparation can be negatively impacted as well.

Another way that the food industry has addressed food preservation is to utilize, as a component in packaging material, an antimicrobial material that directly contacts the food. However, such direct contact may be undesirable in some applications. Many commercial biocides in use are classified as carcinogens, or are toxic to certain organisms. For example, methyl bromide, widely used both in the field and post-harvest to block microbial infections, has been shown to be both toxic and environmentally damaging.

Chlorine dioxide (ClO₂) gas has many properties that make it attractive for treatment of foodstuffs. Studies have shown application of chlorine dioxide can significantly reduce growth of microbes, including Listeria monocytogenes. Chlorine dioxide, sometimes referred to as the “ideal biocide,” has a unique adaptability for use in many forms before and after being added to a package.

Direct exposure of a foodstuff to chlorine dioxide gas can be employed for inhibiting or preventing the growth of microbes; however, the properties that make it appealing for treatment of food reduce its versatility. In particular, chlorine dioxide breaks down rapidly in the atmosphere. This property makes direct treatment with chlorine dioxide gas appealing, since the active agent therefore poses little risk to the consumer. However, the rapid decomposition of chlorine dioxide gas results in the foodstuff becoming vulnerable to post-application contamination.

Many methods for producing chlorine dioxide gas rely on reactions of chemical compounds, e.g., metal chlorite salts. When properly applied, these materials can provide a tunable release of chlorine dioxide gas, including a quick burst if desired for knockdown of existing microbes, followed by a sustained, more dilute application for longer term management of microbes.

Despite popular perception, storage of foodstuffs at reduced temperatures doesn't completely eliminate the threat of microbial contamination, and there remains a need for reducing or eliminating the presence of microbes in frozen foods. Cold storage can be used for produce, including fresh fruits and vegetables, to reduce spoilage from molds, yeasts, and bacteria. Fresh fruits and vegetables are commonly treated in an IQF process. IQF, in the culinary arts and manufacturing systems, stands for “individually quick frozen” and is sometimes referred to as flash freezing. The IQF process is notable due to the positive effects that it has on the fruits and vegetables. The process involves sending the individual food items on a conveyer belt or through a tunnel into a blast chiller that freezes the item almost instantly. Since the items go in separately, they remain separate all the way through the supply chain and into the consumer's hands.

Successful use of chlorine dioxide, and in particular chlorine dioxide releasing agents, for reducing and/or eliminating microbial contamination in frozen foodstuffs is questionable. These reactions not only can be impacted from the decreased rates typical of cold environments, but can also suffer from the absence of a suitable aqueous medium in sub-freezing temperatures that can be necessary for the chlorine dioxide forming reaction. Furthermore, chlorine dioxide gas will condense into a liquid at reduced temperatures. Finally, formation of an ice layer on the surface of frozen foodstuffs can impede exposure of this surface to exterior chlorine dioxide gas.

There remains a need for improved delivery of antimicrobial materials to control, reduce, and substantially destroy microbial contamination in packaged foodstuffs intended for distribution in the frozen state, in particular fresh fruits and vegetables, such as berries, as well as other applications. Such methods should not only eliminate or reduce the microbial load, but also maintain the organoleptic qualities of the foodstuffs, including in particular visual appearance.

### SUMMARY OF THE INVENTION

Accordingly, in one aspect, disclosed herein is a method for inhibiting or preventing the growth of microbes in a powdered or granular or low-moisture and/or densely/closely packed product, the method comprising the steps of:
adding the product to a package suitable for storage of the product;
adding an antimicrobial releasing agent to the package; and
storing the package and the product therein.

Optionally, in any embodiment, the antimicrobial releasing agent is provided in at least one entrained polymer article located within the interior space. The entrained polymer article is a monolithic material that includes a base polymer, the antimicrobial releasing agent and optionally a channeling agent. Preferably, such entrained polymer is provided as a liner or a film.

### BRIEF DESCRIPTION OF THE DRAWINGS

The disclosure will be described in conjunction with the following drawings in which like reference numerals designate like elements and wherein:

FIG. 1 is a perspective view of a plug formed of an entrained polymer that may be deposited onto a substrate according to methods of the disclosed concept.

FIG. 2 is a cross section taken along line 2-2 of FIG. 1.

FIG. 3 is a cross section similar to that of FIG. 2, showing a plug formed of another embodiment of an entrained polymer according to an optional embodiment of the disclosed concept.

FIG. 4 is a schematic illustration of an entrained polymer according to an optional embodiment of the disclosed concept, in which the active agent is an antimicrobial gas releasing material that is activated by contact with a selected material (e.g., moisture).

FIG. 5 is a cross sectional view of a sheet or film formed of an entrained polymer according to an optional embodiment of the disclosed concept, adhered to a barrier sheet substrate.

FIG. 6 is a cross section of a package that may be formed using an entrained polymer according to an optional embodiment of the disclosed concept.

FIG. 7 is a perspective view of an exemplary package incorporating entrained polymer films according to an optional aspect of the disclosed concept.

FIGS. 8(a), 8(b), 8(c), and 8(d) are images that show calorimetry of chlorine dioxide release from entrained polymers, and FIG. 8(e) is a schematic of a mason jar apparatus for testing exposure of a food product to an entrained polymer, in accordance with the invention.

FIGS. 9(a), 9(b), and 9(c) depict a schematic showing time-dependent ice formation during IQF for slow, moderate, and fast freezing, respectively. The four images in each row correspond to increasing time. Scale for the images is minutes. FIGS. 9(d) and 9(e) are images that show IQF samples of brussels sprouts and peas, respectively, in accordance with the invention.

FIGS. 10(a), 10(b), and 10(c) are images depicting flash freezing of raspberries for 15 sec, 30 sec, and 45 sec, respectively. FIGS. 10(d), 10(e), and 10(f) are images showing raspberries subjected to pour-over of liquid N₂, exposure to Dry Ice, and overnight freezing, respectively, in accordance with the invention.

FIGS. 111(a), 11(b), 11(c), and 11(d) are images depicting flash freezing of blueberries for 15 sec, 30 sec, 45 sec, and 60 sec, respectively. FIG. 11(e) is an image showing immersion of blueberries in liquid N₂, in accordance with the invention.

FIGS. 12(a) and 12(b) are images depicting flash freezing of raspberries provided in-house and from a customer, respectively.

FIG. 13 details a procedure for the detection of viral genomes on berries.

FIG. 14 shows mean concentration over time for (a) E. coli (b) Salmonella and (c) L. monocytogenes as log₁₀ CFU/mL neutralized tomato slurry in control trays (i) and treatment trays (ii). Horizontal axis: days; Vertical axis: log₁₀ CFU/mL.

FIG. 15 shows reduction of S. Newport on tomatoes (i) control (ii) InvisiShield. Horizontal axis: days; Vertical axis: log₁₀ CFU/mL.

FIG. 16 shows inactivation of HNV on SS coupons by InvisiShield™ at (a) 0 hr (b) 24 hr (c) 48 hr. Log₁₀ HNV genome equivalent copies (GEC) reductions were calculated by comparing treated coupons to a no treatment control. Dashed line represents the limit of detection of the assay.

FIG. 17 shows log₁₀ reduction of (a) S. enterica (b) E. coli O157:H7 and (c) Listeria monocytogenes on tomatoes at 1, 2, and 7 days.

FIG. 18 shows log₁₀ reduction of (a) S. enterica (b) E. coli O157:H7 and (c) Listeria monocytogenes on blueberries at 1, 2, and 7 days. The dotted line represents the limit of detection of the assay. Different letters indicate significant differences between exposure times (p<0.05).

FIG. 19 shows log₁₀ reduction in genome equivalent copies of HNV in (a) tomatoes and (b) blueberries at 1, 2, and 7 days. The dotted line represents the limit of detection of the assay. Different letters indicate significant differences between exposure times (p<0.05).

FIG. 20 shows log₁₀ reduction in genome equivalent copies of HAV in (a) tomatoes and (b) blueberries at 1, 2, and 7 days. The dotted line represents the limit of detection of the assay. Different letters indicate significant differences between exposure times (p<0.05).

FIG. 21 shows reduction in Microsporidia on tomato slices on exposure to ClO₂ gas. Horizontal axis: hours. Vertical axis: log₁₀ CFU/mL.

FIG. 22 shows growth of (a) Botrytis cinerea (b) Alternaria on blueberries. Horizontal axis: days of refrigerated storage. Vertical axis=log₁₀ cfu/sample.

FIG. 23 shows growth of (a) Botrytis cinerea (b) Alternaria on strawberries. Horizontal axis: days of refrigerated storage. Vertical axis=log₁₀ cfu/sample.

### DETAILED DESCRIPTION OF PREFERRED EMBODIMENTS

While systems, devices and methods are described herein by way of examples and embodiments, those skilled in the art recognize that the systems, devices and methods of the presently disclosed technology are not limited to the embodiments or drawings described. Rather, the presently disclosed technology covers all modifications, equivalents and alternatives falling within the spirit and scope of the appended claims. Any headings used herein are for organizational purposes only and are not meant to limit the scope of the description or the claims.

Disclosed herein is a method for generating ClO₂ gas comprising the step of:
creating an exothermic reaction by contacting an alkali metal chlorite with water under frozen conditions, thereby generating the ClO₂ gas.

Also disclosed herein is a method for inhibiting or preventing the growth of microbes in a quantity of frozen food product, the method comprising the steps of:
storing the quantity of frozen product in a package under frozen conditions; and
generating ClO₂ gas within the package under frozen conditions sufficient to inhibit or prevent the growth of microbes in the quantity of frozen food product.

Also disclosed herein is a method for inhibiting or preventing the proliferation of a virus in a quantity of a product, the method comprising:
providing the quantity of the product in a confined space; and
generating ClO₂ gas in the confined space;
wherein the ClO₂ gas inhibits or prevents the proliferation of a virus in the quantity of the product.

In certain embodiments, the product is susceptible to microbial contamination.

In certain embodiments, the product is chosen from a foodstuff and a non-consumable substance.

In certain embodiments, the product is a food product. In certain embodiments, the product is a frozen food product.

In certain embodiments, the frozen product is susceptible to contamination by one or more microbes. In certain embodiments, the frozen product is a food product. In certain embodiments, the food product is an IQF frozen food product. In certain embodiments, the method further comprises the step of IQF freezing the food product. In certain embodiments, the IQF freezing is accomplished by spraying with a cryogenic liquid. In certain embodiments, the IQF freezing is accomplished by immersing in a cryogenic liquid. In certain embodiments, the cryogenic liquid is liquid N₂. In certain embodiments, the IQF freezing is accomplished by exposure to a cryogenic gas. In certain embodiments, the cryogenic gas is CO₂. In certain embodiments, the IQF freezing is accomplished with a flash freezer.

In certain embodiments, the food product is a fruit or vegetable. In certain embodiments, the fruit or vegetable is chosen from a tomato and a berry. In certain embodiments, the food product is a berry. In certain embodiments, the frozen food product is a berry chosen from any one of raspberry, blueberry, cranberry, boysenberry, gooseberry, huckleberry, gooseberry, elderberry, grape, lingonberry, mulberry, acai berry, blackberry, red currant, and black currant.

In certain embodiments:
the frozen food product is encapsulated in a layer of ice; and
the ClO₂ gas penetrates the layer of ice.

In certain embodiments, the antimicrobial releasing agent is provided in at least one entrained polymer.

In certain embodiments, the microbe is chosen from a bacterium, a virus, and a fungus.

In certain embodiments, the bacterium is chosen from E. coli, Salmonella spp., and Listeria monocytogenes.

In certain embodiments, the virus is chosen from human norovirus and hepatitis A.

In certain embodiments, the fungus is chosen from Microsporidia, Botrytis cinerea, and Alternaria sp.

In certain embodiments, a log₁₀ reduction of 1.0 or better, optionally 2.0 or better, optionally 3.0 or better in microbial load is accomplished by 24 hours. In certain embodiments, a log₁₀ reduction of 1.0 or better, optionally 2.0 or better, optionally 3.0 or better in microbial load is accomplished by 48 hours. In certain embodiments, a log₁₀ reduction of 1.0 or better, optionally 2.0 or better, optionally 3.0 or better in microbial load is accomplished by 72 hours.

In certain embodiments, the entrained polymer comprises a channeling agent.

In certain embodiments, the antimicrobial releasing agent comprises an alkaline metal chlorite. In certain embodiments, the antimicrobial releasing agent comprises sodium chlorite.

In certain embodiments, the base polymer of the entrained polymer ranges from 10% to 70%, optionally from 20% to 60%, optionally from 20% to 50%, optionally from 20% to 40%, optionally from 30% to 70%, optionally from 30% to 60%, from 30% to 50%, optionally from 40% to 70%, optionally from 40% to 60%, optionally from 40% to 50% by weight of the entrained polymer.

In certain embodiments, the antimicrobial releasing agent is in a range from 20% to 80%, optionally 30% to 70%, optionally 30% to 60%, optionally 30% to 50%, optionally from 35% to 70%, optionally from 35% to 60%, optionally from 35% to 55%, optionally from 35% to 50%, optionally 40% to 70%, optionally from 40% to 60%, optionally from 40% to 50%, optionally from 45% to 60%, optionally from 50% to 60% by weight with respect to the total weight of the entrained polymer.

In certain embodiments, the channeling agent is in a range of 1% to 16%, optionally 1% to 14%, optionally from 1% to 12%, optionally from 1% to 10%, optionally from 1% to 8%, optionally from 1% to 6%, optionally from 1% to 5%, optionally from 1% to 4%, optionally from 2% to 16%, optionally from 2% to 14%, optionally from 2% to 12%, optionally from 2% to 10%, optionally from 2% to 8%, optionally from 2% to 6%, optionally from 2% to 5%, optionally from 2% to 4%, optionally from 4% to 12%, optionally from 4% to 10%, optionally from 4% to 8%, optionally from 4% to 6%, optionally from 4% to 5%, optionally from 6% to 12%, optionally from 6% to 10%, optionally from 6% to 8%, optionally from 8% to 12%, optionally from 8% to 10% by weight of the entrained polymer.

In certain embodiments, the entrained polymer is a film. In certain embodiments, the entrained polymer film is exposed to moisture prior to inserting in the package. In certain embodiments, the entrained polymer film is exposed to liquid water prior to inserting in the package.

Also provided are embodiments wherein any embodiment above may be combined with any one or more of these embodiments, provided the combination is not mutually exclusive.

As used herein, two embodiments are “mutually exclusive” when one is defined to be something which is different than the other.

### Definitions

As used herein, the terms below have the meanings indicated.

### List of Abbreviations

EMEM=minimal essential medium; HIFBS=heat inactivated; fetal bovine serum; HAV=hepatitis A; HNV=human norovirus; SS=stainless steel;

As used herein, the term “active” is defined as capable of acting on, interacting with or reacting with a selected material (e.g., moisture or oxygen) according to the disclosure. Examples of such actions or interactions may include absorption, adsorption or release of the selected material. Another example of “active”, which is pertinent to a primary focus of the present disclosure is an agent capable of acting on, interacting with or reacting with a selected material in order to cause a release of a released material, or a bactericidal activity.

As used herein, the term “active agent” is defined as a material that (1) is preferably immiscible with the base polymer and when mixed and heated with the base polymer and the channeling agent, will not melt, i.e., has a melting point that is higher than the melting point for either the base polymer or the channeling agent, and (2) acts on, interacts or reacts with a selected material. The term “active agent” may include but is not limited to materials that absorb, adsorb or release the selected material(s). The active agents of primary focus in this specification are antibacterial releasing agents.

The term “antimicrobial releasing agent” refers to an active agent that is capable of releasing a released antimicrobial material. The released antimicrobial material may be an active component. This active agent may include an active component and other components (such as a carrier) in a formulation (e.g., powdered mixture) configured to release the active component. A “released antimicrobial material” is a compound that inhibits or prevents the growth and proliferation of microbes and/or kills microbes. The released antimicrobial material is released by the antimicrobial releasing agent. By way of example only, an antimicrobial releasing agent may be triggered (e.g., by chemical reaction or physical change) by contact with a selected material (such as moisture). For example, moisture may react with an antimicrobial releasing agent to cause the agent to release a released antimicrobial material.

As used herein, the term “base polymer” is a polymer optionally having a gas transmission rate of a selected material that is substantially lower than, lower than or substantially equivalent to, that of the channeling agent. By way of example, such a transmission rate is a water vapor transmission rate in embodiments where the selected material is moisture and the active agent is an antimicrobial gas releasing agent that is activated by moisture. This active agent may include an active component and other components in a formulation configured to release the antimicrobial gas. The primary function of the base polymer is to provide structure for the entrained polymer.

Suitable base polymers for use in the disclosure include thermoplastic polymers, e.g., polyolefins such as polypropylene and polyethylene, polyisoprene, polybutadiene, polybutene, polysiloxane, polycarbonates, polyamides, ethylene-vinyl acetate copolymers, ethylene-methacrylate copolymer, poly(vinyl chloride), polystyrene, polyesters, polyanhydrides, polyacrylonitrile, polysulfones, polyacrylic ester, acrylic, polyurethane and polyacetal, or copolymers or mixtures thereof.

In certain embodiments, the channeling agent has a water vapor transmission rate of at least two times that of the base polymer. In other embodiments, the channeling agent has a water vapor transmission rate of at least five times that of the base polymer. In other embodiments, the channeling agent has a water vapor transmission rate of at least ten times that of the base polymer. In still other embodiments, the channeling agent has a water vapor transmission rate of at least twenty times that of the base polymer. In still another embodiment, the channeling agent has a water vapor transmission rate of at least fifty times that of the base polymer. In still other embodiments, the channeling agent has a water vapor transmission rate of at least one hundred times that of the base polymer.

As used herein, the term “channeling agent” or “channeling agents” is defined as a material that is immiscible with the base polymer and has an affinity to transport a fluid (liquid or gas phase) substance at a faster rate than the base polymer. Optionally, a channeling agent is capable of forming channels through the entrained polymer when formed by mixing the channeling agent with the base polymer. Optionally, such channels are capable of transmitting a selected material through the entrained polymer at a faster rate than in solely the base polymer.

As used herein, the term “channels” or “interconnecting channels” is defined as passages formed of the channeling agent that penetrate through the base polymer and may be interconnected with each other.

As used herein, the term “entrained polymer” is defined as a monolithic material formed of at least a base polymer, an active agent and optionally also a channeling agent entrained or distributed throughout. An entrained polymer thus are at least two phases (without a channeling agent) or at least three phases (with a channeling agent).

As used herein, the term “monolithic,” “monolithic structure” or “monolithic composition” is defined as a composition or material that does not consist of two or more discrete macroscopic layers or portions. Accordingly, a “monolithic composition” does not include a multi-layer composite.

As used herein, the term “phase” is defined as a portion or component of a monolithic structure or composition that is uniformly distributed throughout, to give the structure or composition its monolithic characteristics.

As used herein, the term “three phase” is defined as a monolithic composition or structure comprising three or more phases. An example of a three phase composition according to the disclosure is an entrained polymer formed of a base polymer, active agent, and channeling agent. Optionally, a three phase composition or structure may include an additional phase, e.g., a colorant, but is nonetheless still considered “three phase” on account of the presence of the three primary functional components.

As used herein, the term “selected material” is defined as a material that is acted upon, by, or interacts or reacts with an active agent and is capable of being transmitted through the channels of an entrained polymer. For example, in embodiments in which a releasing material is the active agent, the selected material may be moisture that reacts with or otherwise triggers the active agent to release a releasing material, such as an active component.

Furthermore, the terms “package,” “packaging” and “container” may be used interchangeably herein to indicate an object that holds or contains a good, e.g., food product and products. Optionally, a package may include a container with a product stored therein. Non-limiting examples of a package, packaging and container include a tray, box, carton, bottle receptacle, vessel, pouch and flexible bag. A pouch or flexible bag may be made from, e.g., polypropylene or polyethylene. The package or container may be closed, covered and/or sealed using a variety of mechanisms including a cover, a lid, lidding sealant, an adhesive and a heat seal, for example. The package or container is composed or constructed of various materials, such as plastic (e.g., polypropylene or polyethylene), paper, Styrofoam, glass, metal and combinations thereof. In one optional embodiment, the package or container is composed of a rigid or semi-rigid polymer, optionally polypropylene or polyethylene, and preferably has sufficient rigidity to retain its shape under gravity.

### Exemplary Entrained Polymers

Conventionally, desiccants, oxygen absorbers and other active agents have been used in raw form, e.g., as loose particulates housed in sachets or canisters within packaging, to control the internal environment of the package. For many applications, it is not desired to have such loosely stored active substances. Thus, the present application provides active entrained polymers comprising active agents, wherein such polymers can be extruded and/or molded into a variety of desired forms, e.g., container liners, plugs, film sheets, pellets and other such structures.

Optionally, such active entrained polymers may include channeling agents, such as polyethylene glycol (PEG), which form channels between the surface of the entrained polymer and its interior to transmit a selected material (e.g., moisture) to the entrained active agent (e.g., desiccant to absorb the moisture). As explained above, entrained polymers may be two phase formulations (i.e., comprising a base polymer and active agent, without a channeling agent) or three phase formulations (i.e., comprising a base polymer, active agent and channeling agent). Entrained polymers are described, for example, in U.S. Pat. Nos. 5,911,937, 6,080,350, 6,124,006, 6,130,263, 6,194,079, 6,214,255, 6,486,231, 7,005,459, and U.S. Pat. Pub. No. 2016/0039955, each of which is incorporated herein by reference as if fully set forth.

Suitable channeling agents in the disclosure include polyglycol such as polyethylene glycol (PEG), ethylene-vinyl alcohol (EVOH), polyvinyl alcohol (PVOH), glycerin polyamine, polyurethane and polycarboxylic acid including polyacrylic acid or polymethacrylic acid. Alternatively, the channeling agent 35 can be, for example, a water insoluble polymer, such as a polypropylene oxide-monobutyl ether, which is commercially available under the trade name Polyglykol B01/240, produced by CLARIANT. In other embodiments, the channeling agent could be a polypropylene oxide monobutyl ether, which is commercially available under the trade name Polyglykol B01/20, produced by CLARIANT, polypropylene oxide, which is commercially available under the trade name Polyglykol DO1/240, produced by CLARIANT, ethylene vinyl acetate, nylon 6, nylon 66, or any combination of the foregoing.

### Entrained Polymer Containing Antimicrobial Agents

Optionally, the antimicrobial releasing agent is a component of an entrained polymer, which is at least two phases and comprises the antimicrobial releasing agent, a base polymer. Optionally, the entrained polymer is at least three phases and comprises the antimicrobial releasing agent, a base polymer, and a channeling agent. The form of the entrained polymer is not limited. Optionally, such entrained polymer is in the form of a film, a sheet, a liner, or a plug.

In general, it is believed that the higher the active agent concentration in the mixture, the greater the absorption, adsorption or releasing capacity (as the case may be) will be of the final composition. However, too high an active agent concentration could cause the entrained polymer to be more brittle, and the molten mixture of active agent, base polymer material and channeling agent to be more difficult to either thermally form, extrude or injection mold.

In any embodiment, the antimicrobial releasing agent loading level or concentration can range from 20% to 80%, optionally 30% to 70%, optionally 30% to 60%, optionally 30% to 50%, optionally from 35% to 70%, optionally from 35% to 60%, optionally from 35% to 55%, optionally from 35% to 50%, optionally 40% to 70%, optionally from 40% to 60%, optionally from 40% to 50%, optionally from 45% to 60%, optionally from 50% to 60% by weight with respect to the total weight of the entrained polymer.

In one embodiment, an entrained polymer may be a three phase formulation including 50% by weight of ASEPTROL 7.05 antimicrobial releasing agent in the form of the powdered mixture, 38% by weight ethyl vinyl acetate (EVA) as a base polymer and 12% by weight polyethylene glycol (PEG) as a channeling agent.

The formulation of the entrained polymer is dictated by the specific components chosen, the desired quantity of active component released, the rate of the desired release of active component, the duration of release, and the total amount of the antimicrobial releasing agent desired for use.

The methods of producing entrained polymers 10 according to the present disclosure are not particularly limited. Examples include blending a base polymer 25 and a channeling agent 35. The active agent 30 is blended into the base polymer 25 either before or after adding the channeling agent 35. All three components are uniformly distributed within the entrained polymer 10 mixture. The entrained polymer thus prepared contains at least three phases.

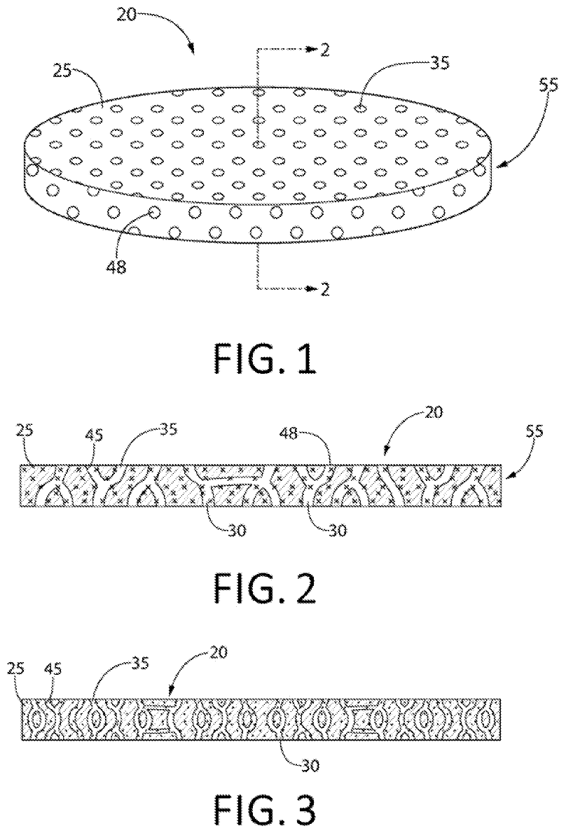

FIGS. 1-6 illustrate entrained polymers 10 and various packaging assemblies formed of entrained polymers according to the disclosure. The entrained polymers 10 each include a base polymer material 25, a channeling agent 35 and an active agent 30. As shown, the channeling agent 35 forms interconnecting channels 45 through the entrained polymer 10. At least some of the active agent 30 is contained within these channels 45, such that the channels 45 communicate between the active agent 30 and the exterior of the entrained polymer 10 via channel openings 48 formed at outer surfaces of the entrained polymer 10. The active agent 30 can be, for example, any one of a variety of absorbing, adsorbing or releasing materials. The term “an active agent” may be understood as “an antimicrobial releasing agent” according to the context. While a channeling agent, e.g., 35, is preferred, the disclosed concept broadly includes entrained polymers that optionally do not include channeling agents.

FIG. 1 shows a plug 55 constructed of an entrained polymer 20, in accordance with certain embodiments of the disclosure. The plug 55 may be placed inside of a container. As aforementioned, the entrained polymer 20 includes a base polymer 25, a channeling agent 35 and an active agent 30.

FIG. 2 shows a cross-sectional view of the plug 55 shown in FIG. 1. In addition, FIG. 2 shows that the entrained polymer 20 has been solidified such that the channeling agent 35 forms interconnecting channels 45 to establish passages throughout the solidified plug 55. At least some of the active agent 30 is contained within the channels 45, such that the channels 45 communicate between the active agent 30 and the exterior of the entrained polymer 20 via channel openings 48 formed at outer surfaces of the entrained polymer 25.

FIG. 3 illustrates an embodiment of a plug 55 having similar construction and makeup to the plug 55 of FIG. 2, where interconnecting channels 45 are finer as compared to those shown in FIG. 2. This can result from the use of a dimer agent (i.e., a plasticizer) together with a channeling agent 35. The dimer agent may enhance the compatibility between the base polymer 25 and the channeling agent 35. This enhanced compatibility is facilitated by a lower viscosity of the blend, which may promote a more thorough blending of the base polymer 25 and channeling agent 35, which under normal conditions can resist combination into a uniform solution. Upon solidification of the entrained polymer 20 having a dimer agent added thereto, the interconnecting channels 45 which are formed there through have a greater dispersion and a smaller porosity, thereby establishing a greater density of interconnecting channels throughout the plug 55.

Interconnecting channels 45, such as those disclosed herein, facilitate transmission of a desired material, such as moisture, gas or odor, through the base polymer 25, which generally acts as a barrier to resist permeation of these materials. For this reason, the base polymer 25 itself acts as a barrier substance within which an active agent 30 may be entrained. The interconnecting channels 45 formed of the channeling agent 35 provide pathways for the desired material to move through the entrained polymer 10. Without these interconnecting channels 45, it is believed that relatively small quantities of the desired material would be transmitted through the base polymer 25 to or from the active agent 30. Additionally, wherein the desired material is transmitted from the active agent 30, it may be released from the active agent 30, for example in embodiments in which the active agent 30 is a releasing material, such as an antimicrobial gas releasing material.

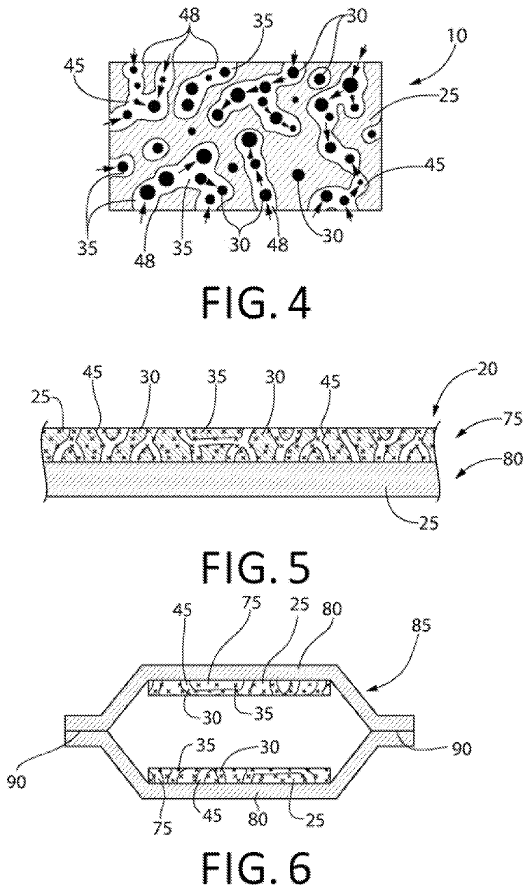

FIG. 4 illustrates an embodiment of an entrained polymer 10 according to the disclosed concept, in which the active agent 30 is an absorbing or adsorbing material. The arrows indicate the path of the selected material, for example moisture or gas, from an exterior of the entrained polymer 10, through the channels 45, to the particles of active agent 30, which absorb or adsorb the selected material.

FIG. 5 illustrates an active sheet or film 75 formed of the entrained polymer 20 used in combination with a barrier sheet 80 to form a composite, according to an aspect of the disclosure. The characteristics of the active sheet or film 75 are similar to those described with respect to the plug 55. The barrier sheet 80 may be a substrate such as foil and/or a polymer with low moisture or oxygen permeability. The barrier sheet 80 is compatible with the entrained polymer structure 75 and is thus configured to thermally bond to the active sheet or film 75, when the active sheet or film 75 solidifies after dispensing.

FIG. 6 illustrates an embodiment in which the active sheet or film 75 and the barrier sheet 80 are combined to form a packaging wrap having active characteristics at an interior surface formed by the entrained polymer 10 in the active sheet or film 75, and vapor resistant characteristics at an exterior surface formed by the barrier sheet 80. In this embodiment, the active sheet or film 75 occupies a portion of the barrier sheet 80. The methods according to the disclosure for making the active sheet or film 75 and adhering it to the barrier sheet 80 are particularly limited.

In one embodiment, the sheets of FIG. 5 are joined together to form an active package 85, as shown in FIG. 6. As shown, two laminates or composites are provided, each formed of an active sheet or film 75 joined with a barrier sheet 80. The sheet laminates are stacked, with the active sheet or film 75 facing one another, so as to be disposed on an interior of the package, and are joined at a sealing region 90, formed about a perimeter of the sealed region of the package interior.

Optionally, in any of the foregoing embodiments, the antimicrobial entrained polymer is in the form of a film that is disposed within a sealed food package. Optionally, the film may be adhered, e.g., using an adhesive, to an inner surface of the package. Alternatively, the film may be heat staked (without an adhesive) to the inner surface of the package. The process of heat staking film onto a substrate is known in the art and described in detail in U.S. Pat. No. 8,142,603, which is incorporated by reference herein in its entirety. Alternatively, the film may be deposited and adhered to the inner surface of the package via a direct in-line melt process. The size and thickness of the film can vary. In certain embodiments, the film has a thickness of approximately 0.2 mm or 0.3 mm. Optionally, the film may range from 0.1 mm to 1.0 mm, more preferably from 0.2 mm to 0.6 mm.

### Frozen Foodstuffs

Certain of the materials and methods disclosed herein are related to the treatment of products, including but not limited to foodstuffs, at reduced temperatures. In particular, disclosed herein is the treatment of frozen products, i.e., products held at a temperature below the freezing point of water. A person of skill will recognize that an object held below the freezing point of water can develop a layer of ice on the surface, and that the amount, and nature, of the layer of ice can be dependent on many factors, including the nature of the object, the ambient humidity, the temperature, and the rate at which the object is cooled to below freezing. Certain objects, under certain conditions, may be coated with a single sheet of ice. Certain objects, under certain conditions, may be coated with a plurality of ice fragments. Certain objects, under certain conditions, may be coated with a layer of small ice crystals, often termed “frost”. All of the aforementioned coatings of frozen water are contemplated with this disclosure.

### Exemplary Containers or Packages According to Disclosure

The entrained polymer containing the antimicrobial releasing agent of the current disclosure may be utilized in packages. The entrained polymer may be attached, adhered, or otherwise included in any container or package via conventional methods. The container or package is used in commerce. The shape or geometry of the container or package is not limited.

In one embodiment, the entrained polymer containing the antimicrobial releasing agent of the current disclosure is in a form of a plug or a sleeve placed in the interior of a container configured for storage of a piece of moist equipment or part. The moisture from the equipment or part activates the antimicrobial releasing agent described above within the entrained polymer and the antimicrobial active component, for example chlorine dioxide, is released from the carrier. The antimicrobial active component thus released prevents and inhibits the bacteria growth within the container or on the moist equipment upon contact.

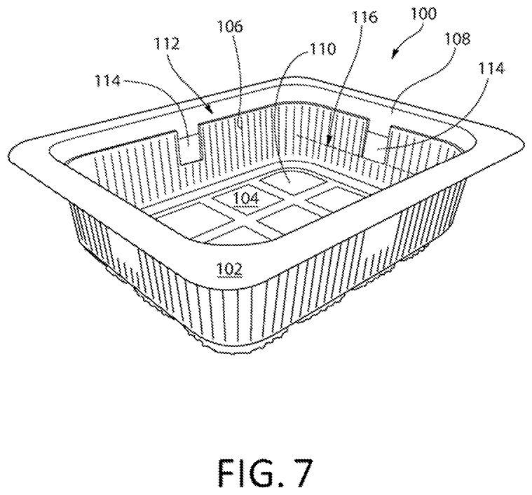

Another embodiment is shown in FIG. 7—a package 100 for storing products, in accordance with certain embodiments of the disclosure. The package 100 is shown in the form of a plastic tray 102. Although, other forms and materials are also contemplated as being within the scope of the disclosure. The tray 102 comprises a base 104, and sidewalls 106 extending vertically from the base 104 leading to a tray opening 108. The base 104 and sidewalls 106 together define an interior 110, e.g. for holding and storing fresh produce. The package 100 optionally includes a flexible plastic lidding film 112, which is disposed over and seals the opening 108. It is contemplated and understood that a wide variety of covers or lids may be used to close and seal the opening 108. Optionally, the cover or lid is transparent, such that the interior can be viewed. When a product (e.g., sliced tomatoes) is stored within the interior 110, empty space surrounding and above the product is herein referred to as “headspace” (not shown).

The package 100 further includes sections of antimicrobial entrained polymer film 114 disposed on the sidewalls 106. In the embodiment shown, there are four sections of such film 114, one section of film 114 per sidewall 106. The film 114 is preferably disposed at or near the top of the sidewall 106, proximal to the opening 108. At least a portion, although preferably most or all of each of the film sections 114 protrude above the midline 116 of the sidewall 106, the midline 116 being centrally located between the base 104 and the opening 108. It has been found that film placement at or towards the top of the package 100 has an effect on efficacy of the film sections 114, as such placement facilitates desirable distribution of released antimicrobial material into the headspace of the package 100. Placing the entrained polymer at too low of a height above the base 104, or beneath the food in the package, has been found not to provide desirable distribution of the released antimicrobial material in the headspace. If mass transfer of the antimicrobial is not optimal, some of the food product/good will not be adequately protected against the growth of microbes. Additionally, the food may undesirably react with and/or absorb the released antimicrobial material. It has been found that placing the film above the midline of the sidewall, preferably at a height of at least 67% or 75% or about 80% of the sidewall, facilitates achieving a desired antimicrobial gas release profile and headspace concentration.

In some embodiments, the package is designed to encourage the producer, distributor, and/or consumer to store the package upright, i.e., with a well-defined top. This can be accomplished by providing a cylindrically shaped package. A further benefit to this shape is that flow of chlorine dioxide gas may be more uniform throughout the cylindrical package than in an oblong package, for which the passage of gas may be substantially different at the junctions between adjacent vertical walls than along the walls themselves.

The package may further be fitted with a removable or openable lid, which can further serve to define the top of the package. In some embodiments, the antimicrobial entrained polymer film is attached to the underside of the removable or openable lid, thereby providing adequate separation between the film and the upper level of the product contained within the package. This orientation may serve to preclude the product from reacting with the released antimicrobial material and/or absorbing this material.

The size of the package is not limited: both large and small packages are contemplated, for storing various quantities of products. In some embodiments, the package may be sufficiently large to provide for a longer term supply of product. In some embodiments, the package may have an appropriate size to contain a supply of product adequate for a shorter period of time, such as 1 week, 2 weeks, 4 weeks, or 8 weeks. Such a package may be suitable for products such as baby formula, which are intended to be fully consumed within a short period, e.g., a month, after initial opening by the consumer. Smaller packages may also provide a more uniform and reproducible delivery of chlorine dioxide gas throughout the entirety of the package.

Optionally, the entrained polymer film 114 is heat staked to the package (e.g., on the sidewall as described and shown vis-à-vis FIG. 7). Advantageously, heat staking could allow the film to permanently adhere to the sidewall without use of an adhesive. An adhesive may be problematic in some circumstances because it may release unwanted volatiles in the food-containing headspace. Aspects of a heat staking process that may be used in accordance with optional embodiments of the disclosure are disclosed in U.S. Pat. No. 8,142,603, as referenced above. Heat staking, in this instance, refers to heating a sealing layer substrate on the sidewall while exerting sufficient pressure on the film and sealing layer substrate to adhere the film to the container wall. Optionally, the entrained polymer film 114 is deposited and adhered to the package via a direct in-line melt adhesion process.

In certain embodiments, the antimicrobial entrained polymer film 114 may be connected to the surface of the lidding film 112 (or a lid) that is inside of the container, in place of the film sections 114 on the sidewall(s) 106, or in addition thereto. Alternatively, the antimicrobial entrained polymer film 114 may be incorporated into the composition of the lidding film 112 (or a lid).

In addition to placement of the film 114, another important factor is the release profile of the released antimicrobial material. As aforementioned, to ensure adequate shelf life, release of the agent must not all occur immediately; rather, release should be extended, sustained and predetermined to attain a desired shelf life.

In general, the polymer entrained with antimicrobial releasing agent is self-activating, meaning that release of the released antimicrobial active component is not initiated until the antimicrobial releasing agent is exposed to the selected material, e.g., moisture. Typically, moisture is not present in the interior, e.g., headspace, of the container prior to a food product being placed inside of the container. Upon placement, the food product generates moisture that interacts with the antimicrobial releasing agent entrained in the polymer, to generate the antimicrobial releasing agent in the container. In one embodiment, the container is sealed in a moisture tight manner to trap moisture within the container generated by moisture-exuding comestibles.

In certain embodiments, a controlled release and/or a desired release profile can be achieved by applying a coating to the active agent, e.g., using a spray coater, wherein the coating is configured to release the released antimicrobial agent within a desired time frame. The antimicrobial releasing agents may have different coatings applied thereon to achieve different release effects. For example, if a 14-day shelf life is desired, based on predetermined relative humidity of the package, the amount of selected material (moisture) present to trigger the antimicrobial releasing agent may be determined. Based on this determination, the agent may be coated with extended release coatings of varying thicknesses and/or properties to achieve the desired release profile. For example, some active agent will be coated such that it will not begin releasing released antimicrobial material until after one week, while other active agent will begin release almost immediately. Spray coating technology is known in the art. For example, pharmaceutical beads and the like are spray coated to control the release rate of active ingredient, e.g., to create extended or sustained release drugs. Optionally, such technology may be adapted to apply coatings to the active agent to achieve a desired controlled rate of release of antimicrobial gas.

Alternatively, a controlled release and/or desired release profile may be achieved by providing a layer, optionally on both sides of the film, of a material configured to control moisture uptake into the entrained polymer (which in turn triggers release of the released antimicrobial material). For example, the film may include a polymer liner, made e.g., from low density polyethylene (LDPE) disposed on either side or both sides thereof. The thickness of the film and liner(s) can vary. In certain embodiments, the film is approximately 0.3 mm thick and the LDPE liners on either side are each approximately 0.02 mm to 0.04 mm thick. The LDPE liners may be coextruded with the film or laminated thereon.

Any combination of the aforementioned mechanisms may be utilized to achieve desired release rates and release profiles of antimicrobial gas within a container.

The invention is further illustrated by the following examples.

### Example 1: Calorimetry

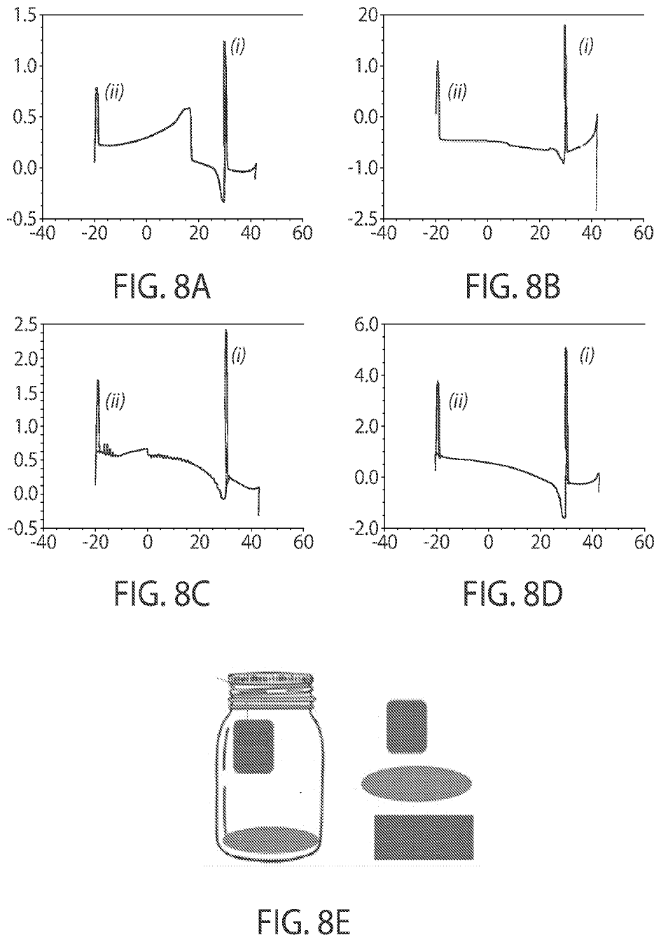

Without wishing to be bound by theory, certain methods as disclosed herein can take advantage of an exothermic reaction for production of chlorine dioxide gas. Towards this goal, calorimetry studies were performed to determine the reaction exotherm. In brief, a 10×15 cm² strip of ActivShield™ film was activated by dipping in water and ramping downward from 40° C. to −20° C. in a calorimeter. Shown in FIG. 8(a)(b)(c)(d) are calorimetry traces corresponding to the production and release of chlorine dioxide gas from the active film. For all samples, an exotherm (i) at approximately 25° C. is observed. The magnitude of this exotherm is: (a) 1.25 W/g; (b) 1.8 W/g; (c) 2.4 W/g; and (d) 5 W/g.

In all cases, an exotherm (i) is observed at approximately −19° C., which is attributed to condensation of the chlorine dioxide gas.

### Example 2: Exposure of Frozen Berries to Active Film

FIG. 8(e) depicts the packaging system utilized for testing of the properties for the entrained polymer. Generally, a test strip of active film (i) was enclosed in a standard tea bag (ii) and suspended in an 1.8 μL mason jar above a 100 g sample of the frozen berry of interest (iii). By this method, direct contact between the frozen berry of interest and the active film was precluded. Prior to enclosure, the active film was dipped in water to activate and placed in the packaging system. All tests were conducted at a temperature between −17° C. to −20° C.

IQF frozen raspberries (100 g/jar) were tested over a range of dosages with ActivShield™ film. The raspberries were exposed to the film for a time period of 60 hours, with a picture taken of every sample at the end of the time period. A percentage of the raspberry sample that exhibited bleaching was determined. See Table 1 for the results of the various dosages tested. Whole raspberries from Nature's Touch (Front Royal, VA) were used in these studies for consistency and to best approximate the typical consumer product.

TABLE 1

| | |
|---|---|
| Amount of Film | % of Raspberries |

| g/lbs.^{(a)} | cm² ^{(b)} | Bleached |
|---|---|---|
| 1.18 | 7.5 | 0 |
| 1.75 | 11.25 | 7.4 |
| 2.35 | 15 | 14.8 |
| 3.13 | 20 | 18.5 |
| 3.91 | 25 | 92.6 |
| 14.08 | 90 | 100 |

^{(a)}g ActivShield ™ film/lbs. of berries
^{(b)} cm² of ActivShield ™ film

Results for studies performed on the scale of 12 raspberries per jar are disclosed in Table 2.

TABLE 2

| | |
|---|---|
| Amount of Film | % of Raspberries |

| g/lbs.^{(a)} | cm² ^{(b)} | Bleached |
|---|---|---|
| 0.5 | 3.14 | 9.16 |
| 1.0 | 6.28 | 21.6 |
| 2.0 | 12.56 | n/d |
| 3.0 | 18.84 | n/d |
| 4.0 | 25.12 | n/d |
| 5.0 | 31.40 | n/d |
| 6.0 | 37.68 | n/d |

^{(a)}g ActivShield ™ film/lbs. of berries
^{(b)} cm² of ActivShield ™ film

It will be evident from the results in Tables 1 and 2 that chlorine dioxide gas is, in fact produced and does, in fact, traverse any existing ice coating to affect the surface of the berry. This test is necessarily non-optimal, in that it makes evident an undesirable side effect of chloride dioxide treatment (i.e. bleaching of the berry) and doesn't reveal the desirable outcome of chlorine dioxide treatment (i.e. reduced microbial load).

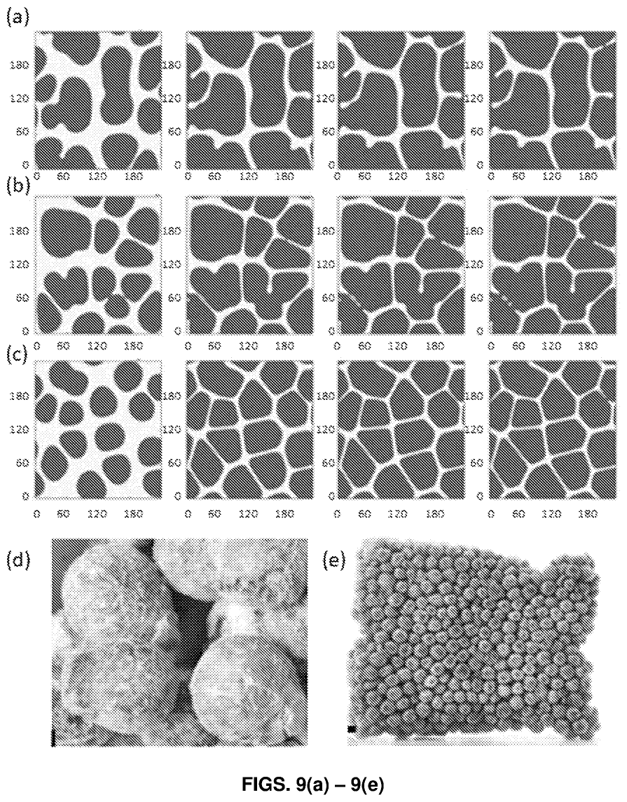

An experiment was conducted to evaluate the effect of the type of ice crystals on the ability of chlorine dioxide to penetrate to the surface of the frozen fruit. In order to do so, a scale was developed to better evaluate the freezing process. The scale is set forth in Table 3. Representative samples are depicted in FIG. 9. The scores and descriptions are provided in Table 3.

TABLE 3

| FIG. | Score | Description |
|---|---|---|
| 2(a) | 1 | Optimal IQF treatment will appear as a |
| | | microscopic sheet or as ice crystals |
| | | with complete coverage. |
| 2(b) | 2 | Separation of berries is clearly visible, |
| | | and ice is in the form of a sheet |
| | | of clearly defined crystals with some separation. |
| 2(c) | 3 | Fairly large ice crystals are seen, |
| | | but some water droplets are also present. |
| 2(d) | 4 | Large ice crystals or distinguishable sheets |
| | | of ice which are similar to freezer burn |
| 2(e) | 5 | The produce presents as one large “iceberg”. |
| | | Organoleptics will be severely compromised |

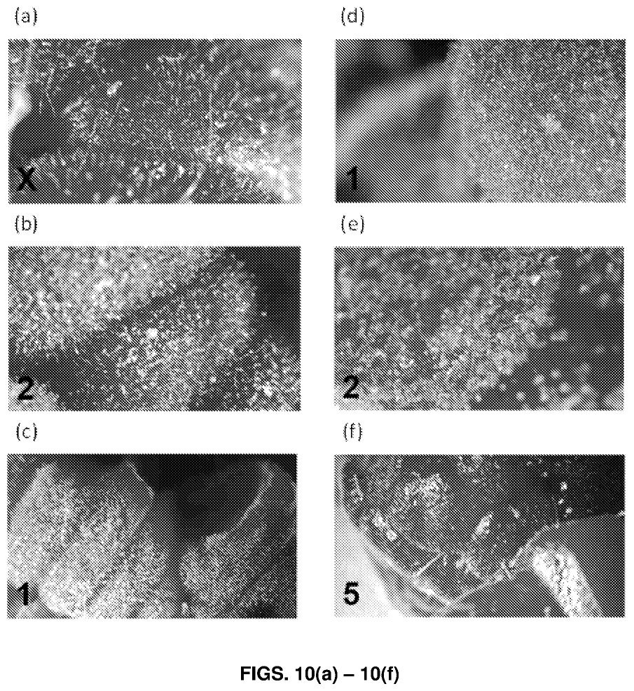

Raspberries were submitted to an IQF design of experiment to determine the rates and times the fruit should be frozen. The anatomy of the raspberry makes it very susceptible to break apart during the freezing process. The droplets are prone to separation at a 1-IQF rating, or if frozen for longer than necessary times. Small quantities of raspberries were frozen by spraying with liquid N₂; larger quantities of raspberries were frozen by pouring liquid N₂ directly over the berries. Results for the two methods were comparable. FIGS. 10(a), 10(b), and 10(c) depict flash freezing of raspberries for 15 sec, 30 sec, and 45 sec, respectively. FIGS. 10(d), 10(e), and 10(f) show raspberries subjected to pour-over of liquid N₂, exposure to Dry Ice, and overnight freezing, respectively. Scores are provided in FIG. 10 for each sample; “X” corresponds to a sample that did not undergo freezing.

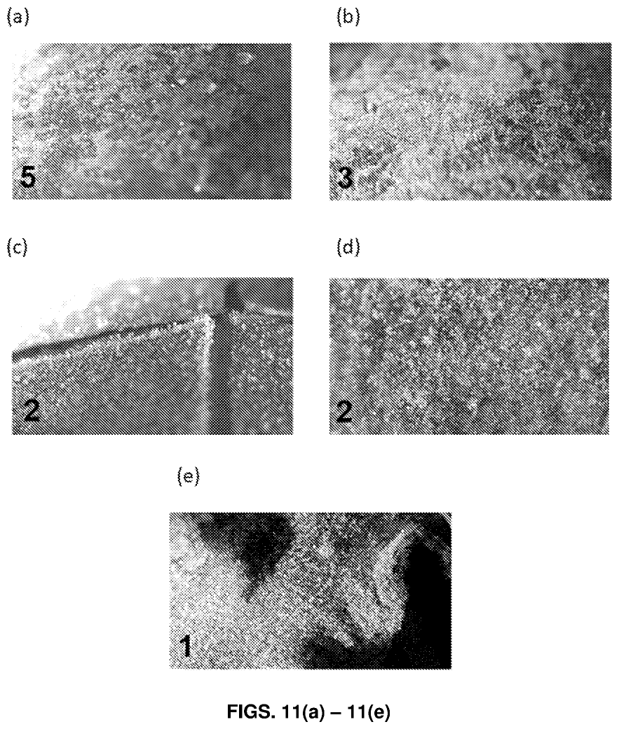

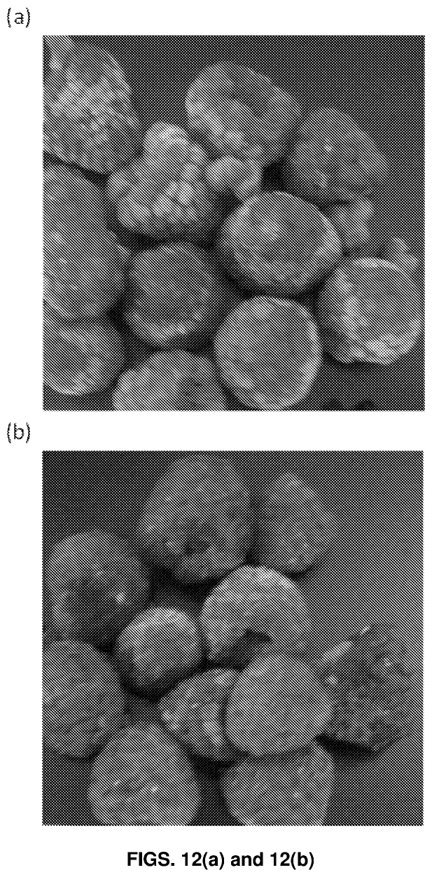

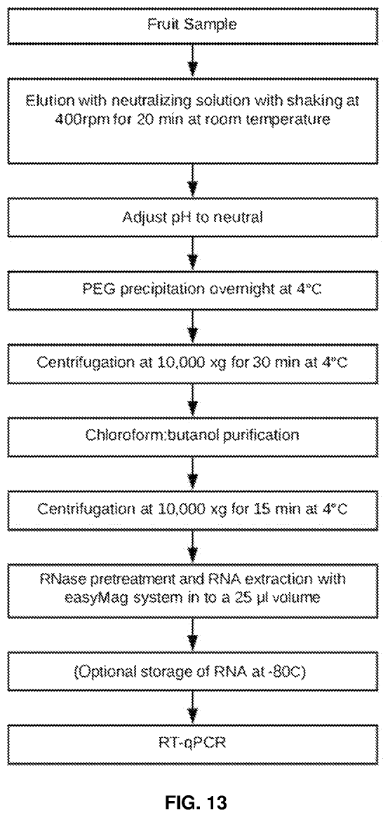

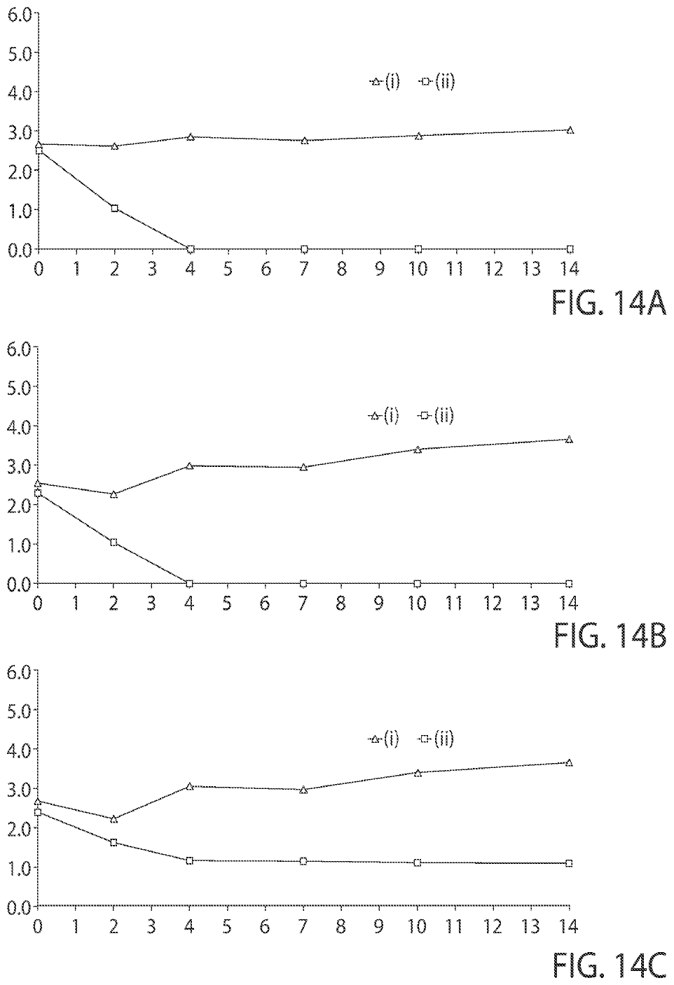

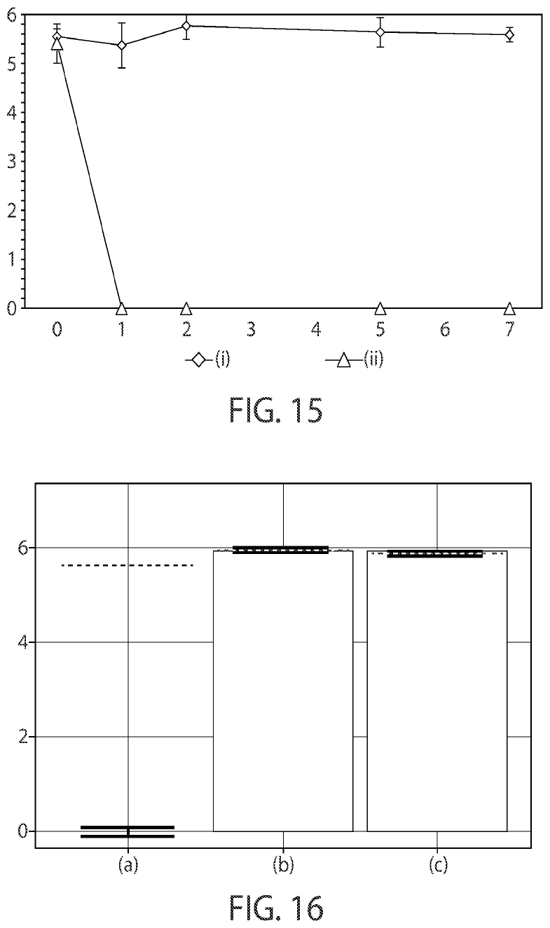

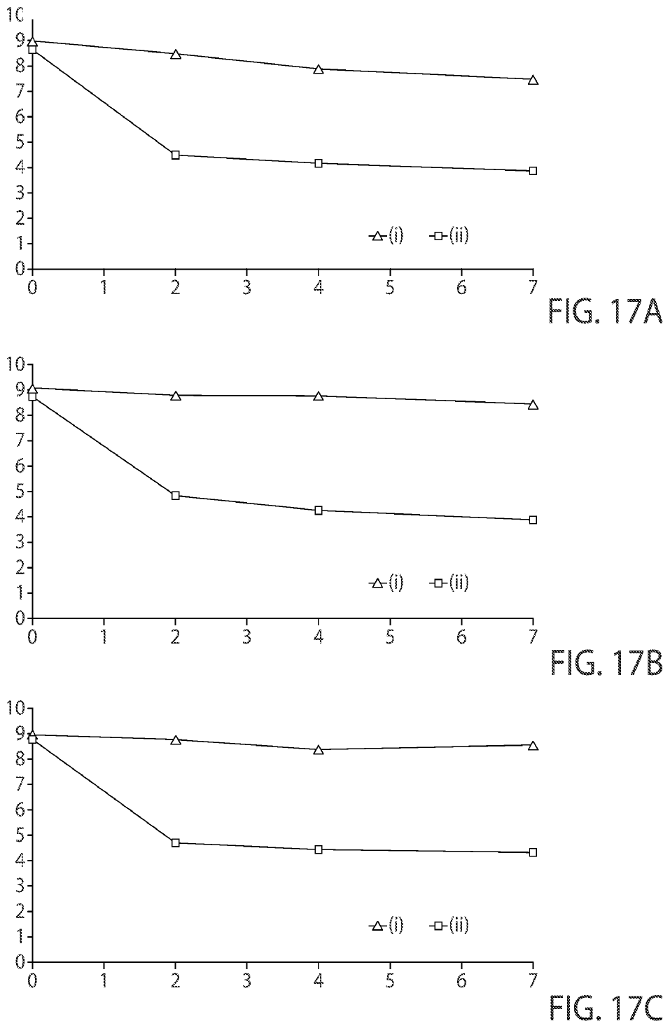

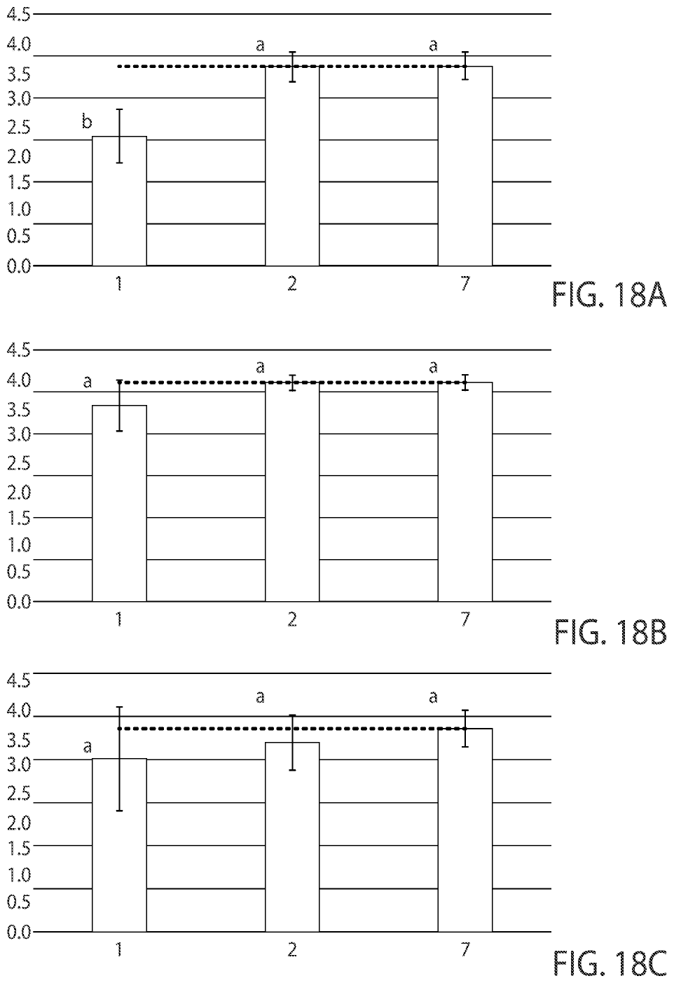

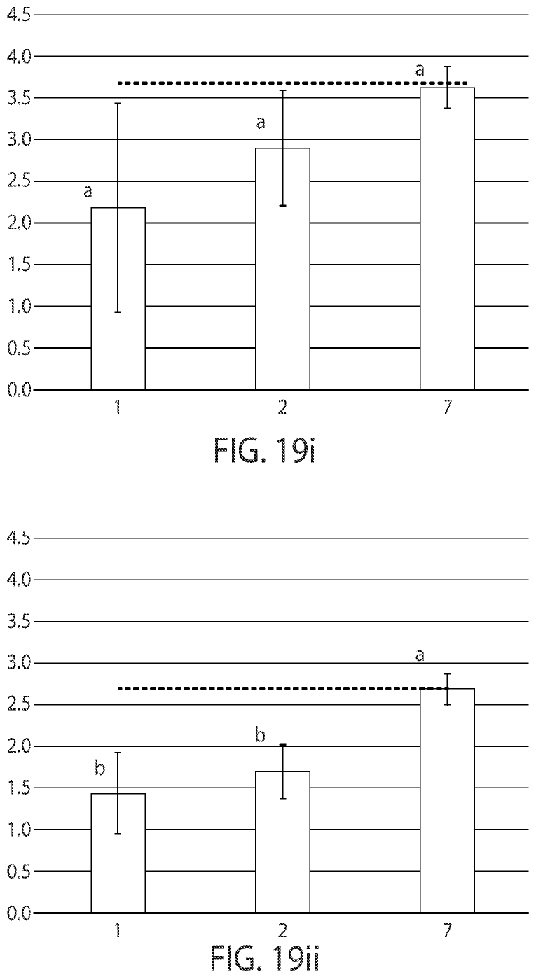

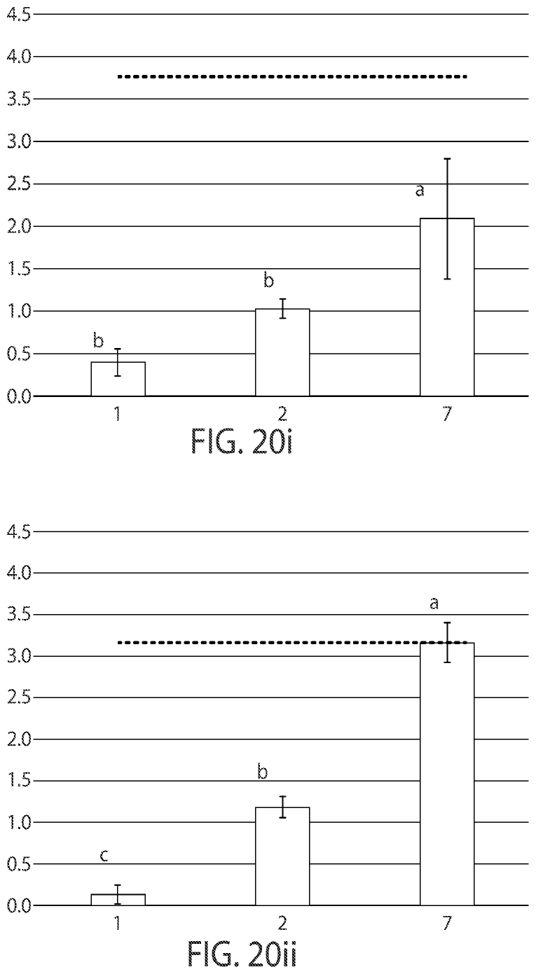

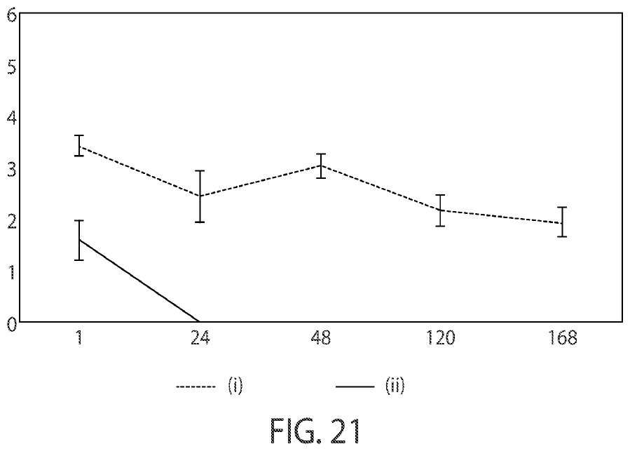

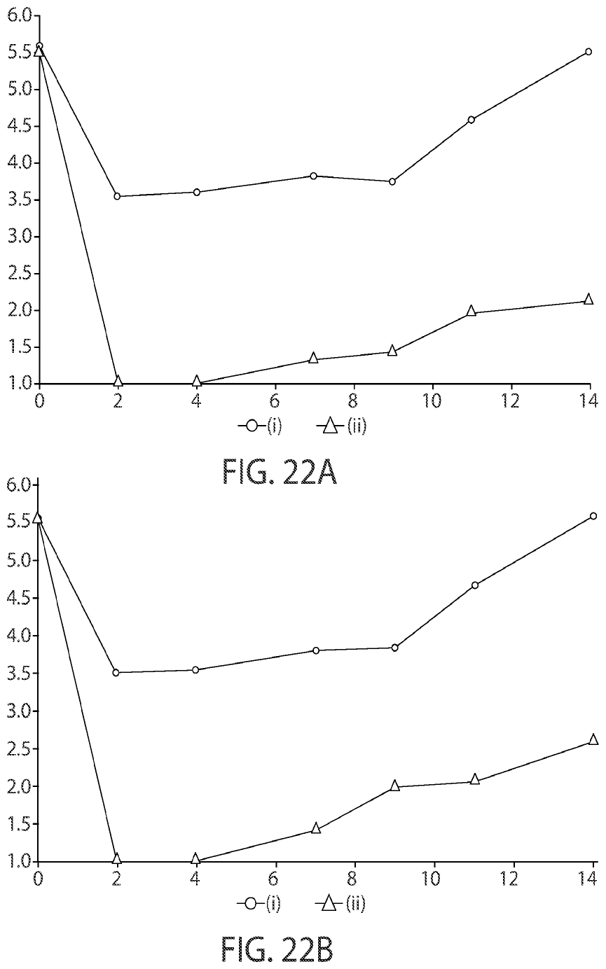

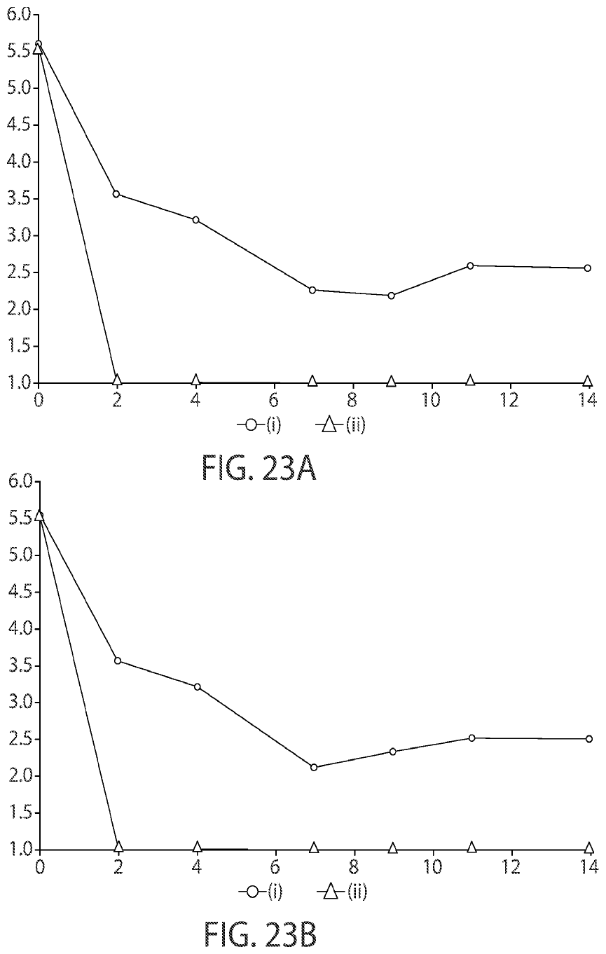

Blueberries were submitted to an IQF design of experiment to determine the rates and times the fruit should be frozen. The surface of this berry's skin is hydrophobic. Due to this property, immersion of blueberries in liquid N₂ is more effective than spraying with liquid N₂. FIGS. 11(a), 11(b), 11(c), and 11(d) depict flash freezing of blueberries for 15 sec, 30 sec, 45 sec, and 60 sec, respectively. FIG. 11(e) shows immersion of blueberries in liquid N₂. Scores are provided in FIG. 11 for each sample.

To further evaluate the IQF technique, a comparison was made between raspberries flash-frozen by in-house methods and by a customer. FIG. 12 depicts the resulting raspberries provided by (a) in-house methods and (b) methods available to a food-processing customer, i.e. end-user. Bleaching of the frozen raspberries is disclosed in Table 4. These results show that the bleaching of frozen raspberries in both cases is comparable.

TABLE 4

| Berry type | Percent bleached | Std Deviation |
|---|---|---|
| In-House | 34 | 1.13 |
| Customer | 32.5 | 0.33 |

### Example 3: Exposure of Frozen Berries to Hepatitis A

The following generally describes materials and methods for determining the effect of InvisiShield treatment on the spread of Hepatitis A virus in IQF blueberries.

Hepatitis A virus (HAV) inoculum consists of strain type HM-175 cell culture lysates obtained from infected FRhK-4 cells treated by multiple freeze thaw cycles. This inoculum will be used directly, without dilution, in all experiments.

Pints of blueberries will be purchased from a local grocery store no more than 3 hours before inoculation. Blueberries will be washed in a 200 ppm chlorine solution for 2 min before being rinsed with sterile deionized water two times. Two blueberries per treatment exposure will each be placed in an aluminum tray and inoculated by pipette with 10 μl of HAV stock (described above) and left to dry at room temperature for 90 min. Inoculated berries will then be stored at 4° C. overnight prior to subjecting them to the ‘mock’ IQF process.

Photographs of the trays of berries will be taken prior to performing the freezing process. “Flash” freezing will be done using liquid N₂, which will be stored in an N₂ dewar and used under containment in a laboratory fume hood. Following overnight refrigerated storage, the inoculated berries, and ten additional berries (not inoculated), will be Individually Quick Frozen (IQF). Briefly, five uninoculated berries will be added to each tray containing a single inoculated berry, and liquid N₂ will be poured over the berries until they are submerged/covered, after which they will be held until the liquid N₂ dissipates (ten to fifteen seconds). The blueberries will be placed in glass jars, and the InvisiShield technology will be pre-wetted, suspended at the top of the jar using dental floss (not touching the frozen product), and the jars sealed. Four concentrations of chlorine dioxide, designed into the novel delivery platform, will be provided, constituting four different treatment doses (1.5, 2, 2.5 and 3 g/lb). Treatment samples will be accompanied by untreated controls (in duplicate). All samples will be held for exposure times of 15 and 30 days. Treatment and control trays will be stored in separate freezers at −15° C. (±2° C.) to prevent confounding by potential outgassing.

Prior to collection of samples for subsequent virus detection, observational notes regarding any negative organoleptic characteristics of the fruit will be taken, along with photos of both the control and treatment trays for side-by-side comparison.

For each experimental timepoint, the two inoculated blueberries per sample will be collected and processed individually for HAV detection. Each piece of fruit will be dropped into a neutralizing solution equal to three times the weight of the sample prior to being processed. To elute any residual virus from the surface of the fruit, the sample will be shaken (400 rpm) in Tris-glycine buffer supplemented with 3% beef extract for 20 min at room temperature. Eluates will be further processed for virus concentration and purification using the EN-ISO-15216 method previously described and summarized (Lowther et al., 2019; Perrin et al., 2015). Prior to RNA extraction, samples will be treated with RNase to eliminate free RNA, allowing the downstream RT-qPCR assay to be more indicative of concentrations of infectious virus. For RNase pre-treatment, 4 μl RNase One (Promega, Madison, WI) and 44 μl reaction buffer will be added to 800 μl of the post-neutralization sample eluate and incubated at 37° C. for 15 min. Samples will be placed on ice for 5 min to abolish RNase enzyme activity prior to RNA extraction, which will be performed using the automated EasyMag system (bioMerieux, Durham, NC) as per manufacturer's instructions. RNA will be eluted in a 25 μl volume of proprietary NucliSENS® elution buffer. HAV RNA will be quantified by RT-qPCR as previously described (Gardner et al., 2003). The concentration of surviving viruses will be determined by extrapolation to an RT-qPCR standard curve generated using RNA transcripts of amplified HAV genome fragments (Manuel et al., 2017).

Three independent replicates will be completed for each of the four treatments, with each replicate being done in duplicate (two containers of two inoculated berries per replicate). Log₁₀ reduction in genome equivalent copy (GEC) will be calculated in comparison to a no treatment control. Results will be expressed as mean±standard deviation of log₁₀ HAV GEC reductions. Results will be compared statistically using ANOVA and the Tukey-Kramer test (Minitab, San Jose, CA). Statistical significance will be established at a level of p<0.05.

### Example 4: Antibacterial Action on Tomatoes

Bacterial strains used in this study are shown in Table 5. Cultures were prepared by streaking stocks on to selective agar (Neogen, Lansing, MI) and incubating for 18-24 h at 35° C., after which single colonies were picked and cultured in 10 mL of Tryptic Soy Broth (TSB; Neogen) for 18-24 h at 35° C. Overnight cultures were then combined in their respective cocktails and used, diluted or undiluted, as inoculum.

TABLE 5
Challenge Bacterial Strains (and selective agars) used in this study

| | | |
|---|---|---|
| E. coli | Salmonella spp. | Listeria monocytogenes |
| (Sorbitol MacConkey Agar) | (Xylose Lysine Desoxycholate | (PALCAM Agar) |
| | Agar) | |
| E. coli O157:H7 | Salmonella Heidelberg | Serotype 2 |
| (ATCC 35150) | (ATCC 8326) | (ATCC 19112) |
| E. coli O111 | Salmonella enteritidis | Serotype 3 |
| (ATCC BAA-2440) | (ATCC 13076) | (ATCC 19113) |
| E. coli O103:H11 | Salmonella typhimurium | Serotype 1/2a |
| (ATCC BAA-2215) | (ATCC 14028) | (ATCC 19111) |
| E. coli O145 | Salmonella typhimurium | Serotype 4b |
| (ATCC BAA-2192) | (ATCC 13311) | (ATCC 19115) |
| E. coli O121:H19 | Salmonella Senftenberg | Serotype 1/2c |
| (ATCC BAA-2219) | (ATCC 43845) | (ATCC 7644) |

Tomatoes were purchased commercially and aseptically sliced. Six slices per replicate were inoculated with 10 μl of the prepared bacterial cocktails to provide a concentration of diluted with TSB to achieve a final concentration of 2-3 log₁₀ CFU/g of sample. Tomatoes were packaged per manufacturer's instructions using InvisiShield™ lidding film or negative control lidding film. Tomato packages were held at 7° C. for 2, 4, 7, 10 and 14 days (n=10).

For bacterial strains, two tomato slices per sample were combined with a volume of Buffered Peptone Water (BPW; 3M, Maplewood, MN) supplemented with 1% Na₂S₂O₃ equal to three times the weight of the sample. This sample was then stomached for 1 min at high speed, after which the neutralized slurry and subsequent serial dilutions in BPW were plated on selective agar (Table 5) and incubated for 48 h at 35° C. Colonies were counted and multiplied by the dilution factor to determine the total log₁₀ CFU/mL remaining in the neutralized slurry (lower limit of detection, 10 CFU/mL neutralized slurry). When no growth was observed, the entirety of the remaining BPW slurry was enriched for 48 h at 35° C. followed by streaking on selective agar (Table 5), and incubated at 35° C. for 48 h. In the absence of typical colonies on selective media, the assay was reported as negative (<1 CFU/mL neutralized slurry). If one or more typical colonies were observed, the sample was reported as positive. A value of <10 CFU/mL of neutralized slurry was used as the detection limit for positive enriched samples. HNV was eluted from the SS coupons by pipetting up and down for approximately two min using 20 μL of PBS, which was then diluted in 180 μL of chlorine neutralizer (1% Na₂S₂O₃). Eluates were processed for detection and enumeration of HNV by RNase pre-treatment, RNA extraction using the NucliSENS EasyMag system (Biomerieux, Durham, NC) and detection by RT-qPCR (Jothikumar, N., J. A. Lowther, K. Henshilwood, D. N. Lees, V. R. Hill, J. Vinjd Appl. Environ. Microb. 2005, 71,1870-1875). Log₁₀ reduction of HNV GEC was determined by comparison to the no treatment control using standard curves.

FIG. 14 shows mean concentration over time for (a) E. coli (b) Salmonella and (c) L. monocytogenes as log₁₀ CFU/mL neutralized tomato slurry in control trays (i) and treatment trays (ii).

For sensory analysis, 42 panelists participated in a triangle test of difference using control and InvisiShield™ treated tomatoes (uninoculated) stored at 4° C. for 3 days. Participants were asked to rate the appearance, flavor and texture of the tomatoes. The data was analyzed by tabulating the number of correct responses and comparing to values in tables for the minimum number of ‘correct’ responses needed to conclude that a perceptible difference existed. For n=42 participants, the number is 22 (a=0.01) (Meilgaard, M., G. V. Civille, and B. T. Carr. 2016. Sensory Evaluation Techniques, CRC Press, Boca Raton, Fla.).

Treated tomatoes were rated as not significantly different in appearance, flavor or texture attributes compared to the control (p<0.05) in the sensory analysis.

### Example 5: Antibacterial Action on Tomatoes

The skins of sliced tomatoes were spot inoculated with S. Newport. Sliced tomatoes were sealed in a controlled environment containing ClO₂ or a regular plastic seal. The inoculated tomato skins were removed and processed on days 0, 1, 2, 5 and 7, after which S. Newport was evaluated by plating onto XLT-4 agar.

S. Newport was grown in TSB at 37° C. for 18-24 hr. Bacteria was resuspended in E-pure water. Serial dilutions of the inoculum were plated onto XLT-4 plates and incubated at 37° C. for 18-24 hours.

Tomatoes were first prepared by rinsing with 70% ethanol and thoroughly rinsing with sterile e-pure water. Tomatoes were sliced using a sterile tomato slicer (Prince Castle, Inc) and placed in pre-labelled trays. Five tomatoes were sliced per Aptar tray (4×10.3×3.4 inches). The tomatoes were spiked by inoculating two slices of each tomato with 10 μL of S. Newport. Tomatoes were incubated at room temperature for 1 hr. After drying, each tray was heat sealed with a tray sealing instrument (Maxwell Chase Technologies) at a temperature of 390° F. For the experimental group, the film was treated with 0.63 g of chlorine dioxide. Trays were stored at 4° C. until specimen collection. For each treatment of 1 hr, 24 hr, 48 hr, and 5 days, and 7 days a control and a treated tray were evaluated. Tomato skins of spiked slices were collected and tested for the presence of S. Newport.

Slices were suspended in peptone water and 2×DE neutralizing broth. The tubes were vortexed and large tomato skins were removed. After removing the tomato skins, the samples were vortexed and serial dilutions were prepared in 0.1% peptone and 100 microliters of sample were plated onto XLT-4 plates. The plates were incubated for 24 hr at 37° C. 100 mL of chlorine dioxide treated sample was also placed in 9 mL of RV broth and stored at 4° C. for 24 hrs.

Reduction in CFU of S. Newport on tomatoes is shown in FIG. 15(i) control (ii) InvisiShield. Consistent with previous research, chlorine dioxide was effective in inactivation of S. Newport, and complete inactivation at two days of treatment with chlorine dioxide.

### Example 6: Antibacterial Action on Tomatoes and Blueberries

Produce was obtained at a local store within 3 h of initiation of experiment. Samples were washed with 200 ppm bleach for 2 min, rinsed 2× with sterile water rinses, then allowed to air dry. Samples were treated with either of three bacterial cultures in TSB:
(a) S. enterica ATCC 4931
(b) E. coli O157:H7 ATCC 43895
(c) L. monocytogenes ATCC 19115

Two treatments and one control per time point were provided. Samples were allowed to dry 1 h, then sealed with either control or treatment films, and stored for various time intervals. Samples were placed in stomacher bags and processed individually. Samples were then eluted with Butterfield's phosphate buffer with 0.1% Na₂S₂O₃. Bacteria were enumerated following serial dilution with PBS. The material was plated on tryptic soy agar and incubated at 37° C. overnight. Colonies were then counted.

Reduction in CFU of (a) S. enterica (b) E. coli O157:H7 and (c) Listeria monocytogenes at 0, 2, 4, and 7 days is shown in FIG. 17 for tomatoes and FIG. 18 for blueberries.

### Example 7: Antiviral Action: SS coupons

A HNV GII.4 Sydney strain obtained as deidentified stool specimen from an outbreak was suspended 20% in PBS with clarification by centrifugation (3,100×g for 5 min at 4° C.). The initial titer of the prepared stock solution was 6-7 log₁₀ genome equivalent copies (GEC) per mL.

Sterile stainless steel (SS) coupons were inoculated with 20 μl of the 20% stool suspension positive for HNV. SS coupons were packaged per manufacturer's instructions using InvisiShield™ lidding film or negative control lidding film. SS packages were held at room temperature for 1 and 2 days (n=3).

HNV was eluted from the SS coupons by pipetting up and down for approximately 2 min using 20 μL of PBS, which was then diluted in 180 μL 1% Na₂S₂O₃. Eluates were processed for detection and enumeration of HNV by RNase pre-treatment, RNA extraction using the NucliSENS EasyMag system (Biomerieux, Durham, NC) and detection by RT-qPCR (1). Log₁₀ reduction of HNV GEC was determined by comparison to the no treatment control using standard curves.

Inactivation of HNV on SS coupons is shown in FIG. 16.

### Example 8: Antiviral Action on Tomatoes and Blueberries

Blueberries and grape tomatoes were purchased commercially no more than 3 hours prior to inoculation. They were washed in a 200 ppm bleach solution for 2 min, rinsed twice with sterile deionized water, and allowed to dry. Twelve tomatoes or twelve blueberries were placed in trays supplied by Aptar CSP Technologies (Atlanta, GA) and two tomatoes or two blueberries per tray were spot inoculated by adding a 10 μl volume of the challenge virus suspension per fruit item.

Samples were treated with either of the following frozen virus stock:
(a) HNV 20% stool suspension
(b) HAV 20% cell lysate suspension

One of the inoculated samples was placed in the middle of the tray, and the second at the outer edge of the tray. A total of two trays of samples were prepared for each replicate for each sampling time point, and uninoculated controls as well as no treatment controls were included for each replicate/time point. Produce was packaged per manufacturer's instructions using InvisiShield™ lidding film or negative control lidding film. Packages were held at 7° C. for 0 (control), 24 or 48 hrs, and 7 days (n=3). Treatment and control trays were stored in separate locations to prevent confounding by potential outgassing.

The two inoculated tomatoes or blueberries per sample were processed individually. Each sample was combined with Tris-base glycine beef extract (3% in sterile deionized water) neutralizing solution equal to three times the weight of the sample and the viruses eluted by shaking at 400 rpm for 20 min at room temperature in filter stomacher bags. Eluate solutions were centrifuged at 10,000×g for 30 min at 4° C. to sediment debris, followed by polyethylene glycol precipitation (12% PEG) overnight at 4° C. Virus-laden precipitates were collected by centrifugation at 10,000×g for 30 min at 4° C.

Sample concentrates were pretreated with RNase prior to RNA extraction using the automated EasyMag system (bioMerieux, Durham, NC) as per manufacturer instructions. RNA was amplified by RT-qPCR targeting the conserved ORF1-ORF2 junction of HNV (Jothikumar et al., 2005) and for the highly conserved 5′ noncoding region of HAV (Costafreda, Bosch & Pint6, 2006). For quantification, the resulting cycle threshold (CT) values were compared to a standard curve produced by serial dilutions of viral RNA obtained from the inoculum. Reduction in GEC as a function of treatment was calculated by subtracting the remaining virus log₁₀ GEC for each treatment from that obtained for the no treatment control.

Each experiment was repeated in triplicate or quadruplicate. Results were expressed as mean+standard deviation of log₁₀ GEC reduction for each product. Data were compared statistically using ANOVA and the Tukey-Kramer test (Minitab Statistical Software, State College, PA). Statistical significance was established at a level of p<0.05.

Reduction in CFU of HNV on (a) tomatoes and (b) blueberries at 0, 2, 4, and 7 days is shown in FIG. 19.

Reduction in CFU of HAV on (a) tomatoes and (b) blueberries at 0, 2, 4, and 7 days is shown in FIG. 20.

Log₁₀ reductions (LR) in HNV GEC for tomatoes were 2.2±1.3, 2.9±0.7, and 3.6±0.3, after 24, 48 hrs and 7 days, respectively (FIG. 19(a)). For blueberries, LR in HNV GEC were 1.4±0.7, 1.7±0.5, and 2.7±0.2, after 24, 48 hrs and 7 days, respectively (FIG. 19(b)). For both product types, efficacy against HNV continued to increase at each time point and reached the LOD at 7 days. However, there were no statistically significant differences in log₁₀ HNV GEC reduction on tomatoes when comparing exposure times. For blueberries, there was significantly greater reduction in log₁₀ HNV GEC after 7 days compared to 24 and 48 hrs. Position of the fruit in the tray did not affect inactivation.

Log₁₀ reductions (LR) in HAV GEC for tomatoes were 0.4±0.2, 1.0±0.1, and 2.1±0.7, after 24 hrs, 48 hrs and 7 days, respectively (FIG. 20(a)). Efficacy against HAV continued to increase at each time point but never reached the limit of detection. There was significantly greater reduction in log₁₀ HAV GEC after 7 days compared to 24 and 48 hrs. HAV GEC LR for blueberries were 0.1±0.2, 1.2±0.4, and 3.2±0.2, after 24 hrs, 48 hrs and 7 days, respectively and efficacy reached the LOD at 7 days (FIG. 20(b)). There was significantly greater reduction in log₁₀ HAV GEC after 7 days compared to 48 hrs, and at 48 hrs compared to 24 hours. Position of the fruit in the tray did not affect inactivation.

### Example 9: Antifungal Action on Tomatoes

Rabbit kidney (Rk-13) cell lines were maintained in flasks using 3.5% Fetal Bovine Serum (HIFBS) EMEM medium 1× antibiotics and antimycotics. Flasks were incubated at 37° C. and 5% CO₂ until preparation of slides. 16-microwell slides were seeded with cells and grown in 10% (HIFBS) EMEM medium for 48 hours before inoculation with spores.

Fresh Microsporidia spores were propagated and harvested using RK-13 tissue cultures. The harvested medium containing cell debris and spores was filtered through a 5 μm filter, centrifuged at 2000 g for 15 minutes and counted using a hemocytometer.

Tomatoes were first prepared by rinsing with 70% ethanol and thoroughly rinsing with sterile e-pure water. Tomatoes were sliced using a sterile tomato slicer (Prince Castle, Inc) and placed in pre-labelled trays. Five tomatoes were sliced per Aptar tray (4×10.3×3.4 inches). The tomatoes were spiked by inoculating two slices of each tomato with 10 microliters of oocysts. Tomatoes were incubated at room temperature for 1 hr. After drying, each tray was heat sealed with a tray sealing instrument (Maxwell Chase Technologies) at a temperature of 390° F. For the experimental group, the film was treated with 0.63 g of chlorine dioxide. Trays were stored at 4° C. until specimen was collection. For each treatment of 1 hr, 24 hr, 48 hr, and 5 days, and 7 days a control and a treated tray were evaluated. Tomato skins of spiked slices were collected and tested for the presence of Microsporidia.

Tomato slices spiked with Microsporidia were suspended in F12 media. The tubes were vortexed and large tomato skins were removed. The Microsporidia samples were centrifuged at 5000 rpm for 5 m. After three washes spores were resuspended in 3.5% (HIFBS) EMEM medium. Serial dilutions were prepared, and tissue culture microwells containing the RK-13 monolayers were inoculated with the sample. The slides were incubated at 35° C. for 3 to 5 days. The glass slides were removed, and the cells were fixed using 100% methanol for 10 minutes, stained with Calcofluor White for 3 min and rinsed with e-pure water. Spore counts were taken using an epifluorescence microscope at 40× magnification and recorded.

Microsporidia spores were easily identified using Calcofluor White stain and evaluation under a UV microscope. There was a clear visual difference between spore viability of the non-treated samples as compared to the chlorine dioxide treated samples. Results show that chlorine dioxide treatment via InvisiShield™ Modified Atmosphere Technology effectively reduced spore viability after one hour of treatment. Spore viability was completely inactivated following 24 hours of treatment with chlorine dioxide.

FIG. 21 shows reduction in Microsporidia on tomatoes.

### Example 10: Antifungal Action on Berries

Sealing equipment, lidding film, and product trays (⅙ steam tray including the InvisiShield™ treatment or an untreated control) were provided by Aptar Food+Beverage-Food Protection.

Botrytis cinerea (ATCC #11542) and Alternaria sp. (ATCC #20084) challenge organisms were prepared for this study. Each culture was prepared from a lyophilized preparation according to manufacturer's instructions or from stock plates. Cultures were transferred to Potato Dextrose Agar (PDA, Hardy Diagnostics, Santa Maria, CA) and incubated at 25±2° C. for 5 days. After incubation, cultures were harvested from the surface of the agar using sterile diluent. The resulting fungal suspensions were used as the inocula below.

Samples of blueberries and sliced strawberries were purchased commercially by Analytical Food Laboratories (AFL). packaged in trays (with or without an antimicrobial), and surface inoculated with Botrytis cinerea and Alternaria spp. immediately upon packaging by adding a volume of each challenge organism suspension to the sample surface. The combined inoculum was dispersed throughout a full tray of product (approximately 1.25 lbs. per tray). The target concentration for both matrices was approximately 105 cfu/sample. Inoculated trays of samples were prepared, including InvisiShield™ test trays and control trays for both blueberries and strawberries. An additional uninoculated tray of each type of berry was also prepared.

Samples were held at refrigerated temperatures. At predetermined storage intervals, samples were evaluated for the amount of each challenge organism remaining. The results were used to determine the effect of the antimicrobial treatment in each matrix (blueberries and sliced strawberries).

Post-inoculation, one control tray, one InvisiShield™ test tray, and one uninoculated control tray for each berry type was sampled for Day 0 evaluation. The remaining trays were placed in refrigerated storage at 7° C., and one control tray and three InvisiShield™ test trays per berry type were sampled after 2, 4, 7, 9, 11, and 14 days of storage.

Three replicate samples from each tray (25 g each) were combined with a volume of sterile diluent (Butterfield's Phosphate Buffer or equivalent) equal to a 1:10 dilution of the sample. The sample was stomached for up to 30 seconds at high speed. Samples were spread plated at appropriate dilutions on PDA agar and incubated at 25±2° C. for 5 days. After incubation, plates were enumerated using a Quebec colony counter. The number of observed colonies for each challenge organism was multiplied by the dilution factor to determine the total count in cfu/sample.

The raw count observed for each sample was converted to log₁₀ cfu/sample. The amount of each challenge organism present at each testing interval was compared to the amount present in the initial samples to determine the ability of the antimicrobial delivered by InvisiShield™ technology to control the outgrowth of each organism in each matrix.

FIG. 22 shows growth of (a) Botrytis cinerea (b) Alternaria on blueberries. Horizontal axis: days of refrigerated storage. Vertical axis=log₁₀ cfu/sample.

FIG. 23 shows growth of (a) Botrytis cinerea (b) Alternaria on strawberries. Horizontal axis: days of refrigerated storage. Vertical axis=log₁₀ cfu/sample.

In the blueberry samples, InvisiShield™ technology reduced both challenge organisms over the first four days of refrigerated storage, followed by a period of recovery. FIG. 22(a) illustrates the reduction of Botrytis growth in blueberries stored in the control container versus an InvisiShield™ treatment tray. The blueberries in the InvisiShield tray showed a reduction of 4.5 logs when sampled at days two and four versus the control tray's reduction of 2 logs. The InvisiShield treatment tray kept growth of Botrytis low through day 14, ultimately resulting in a 3.5 log difference between the blueberries in the treatment tray versus the control tray.

The immediate reduction and recovery behaviors related to Alternaria growth on blueberries in the InvisiShield™ and control trays paralleled the results with Botrytis (FIG. 22(b)).

In the strawberry samples, both inocula were heavily reduced (FIG. 23), even in the control samples. However, both inocula were suppressed below the level of detection using the InvisiShield™ system. Minimal non-specific fungal organisms were detected in both types of berries, at an average value of 27 cfu/g for the blueberries, and 30 cfu/g for the strawberries.

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

1. A method for generating ClO₂ gas comprising the step of:
creating an exothermic reaction by contacting an alkali metal chlorite with water under frozen conditions, thereby generating the ClO₂ gas.

2. The method of claim 1, wherein the ClO₂ gas is generated in a confined space.

3. The method of claim 2, wherein the alkali metal chlorite is provided in an entrained polymer.

4. The method of claim 3, wherein:
the entrained polymer comprises a base polymer chosen from polypropylene, polyethylene, polyisoprene,
polybutadiene, polybutene, polysiloxane, polycarbonates, polyamides, ethylene-vinyl acetate copolymers, ethylene-methacrylate copolymer, poly(vinylchloride), polystyrene, polyesters, polyanhydrides, polyacrylonitrile, polysulfones, polyacrylic ester, acrylic, polyurethane, polyacetal, and copolymers or mixtures thereof; and
the base polymer ranges from 10% to 90% by weight of the total composition.

5. The method of claim 4, wherein:
the entrained polymer further comprises a channeling agent chosen from polyethylene glycol (PEG), ethylene-vinyl alcohol (EVOH), polyvinyl alcohol (PVOH), glycerin polyamine, polyurethane, polyacrylic acid and polymethacrylic acid; and
the channeling agent is provided in a range of 2% to 15% by weight.

6. The method of claim 2, wherein the confined space contains a frozen product susceptible to contamination by one or more microbes, and wherein the ClO₂ gas is provided in an amount sufficient to inhibit or prevent the growth of at least one of the one or more microbes.

7. (canceled)

8. (canceled)

9. The method of claim 6, wherein the frozen product is a food product.

10. The method of claim 9, wherein the food product is an IQF frozen food product.

11. (canceled)

12. The method of claim 10, further comprising the step of IQF freezing the food product wherein the IQF freezing by comprises a process selected from spraying with a cryogenic liquid, immersing in a cryogenic liquid, exposing to a cryogenic gas, and utilizing a flash freezer.

13. (canceled)

14. (canceled)

15. (canceled)

16. (canceled)

17. (canceled)

18. The method of claim 9, wherein:
the food product is encapsulated in a layer of ice; and
the ClO₂ gas penetrates the layer of ice.

19. (canceled)

20. The method of claim 18, wherein the frozen food product is chosen from any one of raspberry, blueberry, cranberry, boysenberry, gooseberry, huckleberry, gooseberry, elderberry, grape, lingonberry, mulberry, agai berry, blackberry, red currant, and black currant.

21. A method for inhibiting or preventing the growth of microbes in a quantity of frozen food product, the method comprising the steps of:
storing the quantity of frozen food product in a package under frozen conditions; and
generating ClO₂ gas within the package under frozen conditions sufficient to inhibit or prevent the growth of microbes in the quantity of frozen food product.

22. The method of claim 21, wherein the ClO₂ gas is obtained by contacting an alkali metal chlorite with water, and wherein the alkali metal chlorite is provided in an entrained polymer.

23. (canceled)

24. The method of claim 22, wherein:
the entrained polymer comprises a base polymer chosen from polypropylene, polyethylene, polyisoprene, polybutadiene, polybutene, polysiloxane, polycarbonates, polyamides, ethylene-vinyl acetate copolymers, ethylene-methacrylate copolymer, poly(vinylchloride), polystyrene, polyesters, polyanhydrides, polyacrylonitrile, polysulfones, polyacrylic ester, acrylic, polyurethane, polyacetal, and copolymers or mixtures thereof;
and the base polymer ranges from 10% to 90% by weight of the total composition.

25. The method of claim 24, wherein:
the entrained polymer further comprises a channeling agent chosen from polyethylene glycol (PEG), ethylene-vinyl alcohol (EVOH), polyvinyl alcohol (PVOH), glycerin polyamine, polyurethane and polycarboxylic acid including polyacrylic acid and polymethacrylic acid; and
the channeling agent is provided in a range of 2% to 15% by weight.

26. (canceled)

27. The method of claim 25, further comprising the step of IQF freezing the food product selected from the group consisting of contacting the food product with cryogenic liquid, and flash freezing.

28. (canceled)

29. (canceled)

30. The method of claim 27, wherein the food product is a berry chosen from any one of raspberry, blueberry, cranberry, boysenberry, gooseberry, huckleberry, gooseberry, elderberry, grape, lingonberry, mulberry, agai berry, blackberry, red currant, and black currant.

31. A method for inhibiting or preventing the proliferation of a virus in a quantity of a product, the method comprising:
providing the quantity of the product in a confined space; and
generating ClO₂ gas in the confined space;
wherein the ClO₂ gas inhibits or prevents the proliferation of a virus in the quantity of the product.

32. The method of claim 31, wherein the virus is chosen from human norovirus and Hepatitis A virus.

33. The method of claim 31, wherein the product is a food product.

34. The method of claim 33, wherein the ClO₂ gas is obtained by contacting an alkali metal chlorite with water, and wherein the alkali metal chlorite is provided in an entrained polymer.

35. The method of claim 34, wherein:
the entrained polymer comprises a base polymer chosen from polypropylene, polyethylene, polyisoprene,
polybutadiene, polybutene, polysiloxane, polycarbonates, polyamides, ethylene-vinyl acetate copolymers, ethylene-methacrylate copolymer, poly(vinylchloride), polystyrene, polyesters, polyanhydrides, polyacrylonitrile, polysulfones, polyacrylic ester, acrylic, polyurethane, polyacetal, and copolymers or mixtures thereof; and
the base polymer ranges from 10% to 90% by weight of the total composition.

36. The method of claim 35, wherein:
the entrained polymer further comprises a channeling agent chosen from polyethylene glycol (PEG), ethylene-vinyl alcohol (EVOH), polyvinyl alcohol (PVOH), glycerin polyamine, polyurethane, polycarboxylic acid polyacrylic acid and polymethacrylic acid; and
the channeling agent is provided in a range of 2% to 15% by weight.

37. The method of claim 33, wherein the package and the food product therein are stored under refrigerated conditions, and wherein the food product is selected from a fruit and a vegetable.

38. (canceled)

39. (canceled)

40. (canceled)

41. The method of claim 37, wherein a log₁₀ reduction of 1.0 or better in microbial load is accomplished by 24 hours.

42. The method of claim 37, wherein a log₁₀ reduction of 2.0 or better in microbial load is accomplished by 7 days.
